(19) United States
(12) Patent Application Publication
(10) Pub. No.: US 2024/0299890 A1
(43) Pub. Date: Sep. 12, 2024

(54) HELICAL MIXER

(71) Applicant: Radiometer Medical ApS, Brønshøj (DK)

(72) Inventors: Peter Emil LARSEN, Brønshøj (DK); Willy Lindegaard ANDERSEN, Brønshøj (DK)

(21) Appl. No.: 18/258,432

(22) PCT Filed: Dec. 22, 2021
(86) PCT No.: PCT/EP2021/087415

§ 371 Date: Jun. 20, 2023

(30) Foreign Application Priority Data
Dec. 22, 2020 (EP) 20216612.0

(51) Int. Cl. B01F 25/433; B01F 25/51; B01F 35/221; C08L 27/12; G01N 1/38

(52) U.S. Cl. CPC B01F 25/4331; B01F 25/51; B01F 35/2215; C08L 27/12; G01N 1/38

(57) ABSTRACT

A helical mixer for a blood analyzer is disclosed. The blood analyzer comprises an inlet configured to receive a blood sample and a reagent. The blood analyzer comprises an outlet configured to output a mixture of the blood sample and the reagent. The blood analyzer comprises a helical flow path extending between the inlet and the outlet. The helical mixer is configured to mix the blood sample with the reagent.

### FIELD

The present disclosure relates generally to the field of hematology. More particularly, it relates to helical mixers, such as for blood analyzers and/or hematological analysis.

### BACKGROUND

Hematology is a branch of medicine covering diseases related to the blood and its components, including methods of treatment, diagnosis, analysis, etc. Hematology encompasses a number of different assessments that can be performed on blood and/or components of the blood. One or more of the assessments could require preparation of a blood sample prior to the actual assessment.

Typical hematology analysis is performed in a laboratory setting, and requires transportation of a blood sample to the laboratory. Thus, there is significant time delay between receiving a blood sample and providing analysis, which can slow down necessary patient care.

### SUMMARY

Accordingly, there is a need for accurate and fast hematology analysis in order to quickly assess and analyze components of a patient's blood. In particular, there is a need for accurate and fast point-of-care hematology analysis.

It is an object of some embodiments to solve or mitigate, alleviate, or eliminate at least some of the above or other disadvantages.

A first aspect is a helical mixer for a blood analyzer. The helical mixer can include an inlet. The inlet can be configured to receive a blood sample. The inlet can be configured to receive a reagent. The inlet can be configured to receive a blood sample and a reagent. The helical mixer can include an outlet. The outlet can be configured to output a mixture of the blood sample and the reagent. The helical mixer can include a helical flow path. The helical flow path can be extending between the inlet and the outlet. The helical mixer can be configured to mix the blood sample with the reagent.

A second aspect is a method of preparing a blood sample. The method can include providing a blood sample and a reagent into an inlet. The method can include mixing the blood sample and the reagent via translating the blood sample and the reagent through a helical flow path in communication with the inlet. The method can include outputting the mixed blood sample and the reagent at an outlet in communication with the inlet.

A third aspect is a blood analyzer. The blood analyzer comprises an inlet module; one or both of a blood gas sensor device and a blood count sensor device; and an output. The blood analyzer may comprise a helical mixer as disclosed herein.

A fourth aspect is a blood analyzer for analyzing multiple blood parameters. The blood analyzer comprising an inlet module configured to receive an initial blood sample; optionally a blood gas sensor device in fluid communication with the inlet module, the blood gas sensor device configured to receive a first blood sample of the initial blood sample and conduct a blood gas analysis on the first blood sample to determine a blood gas analysis parameter; a blood count sensor device in fluid communication with the inlet module, the blood count sensor device configured to receive a second blood sample of the initial blood sample and determine a blood count parameter; and an output configured to provide the blood gas analysis parameter and the blood count parameter.

It is an important advantage of the present disclosure to in a fast and reliable manner be able to mix a blood sample and a reagent together. In particular, it is an advantage of the present disclosure avoid destroying blood cells, e.g. to not activate platelets and/or white blood cells in the blood sample while mixing the reagent with the blood sample. Activating the blood sample can lead to clumping, or other consolidation, which negatively affects analysis. Further, an advantage of the present disclosure is not separating particles of the blood sample during mixing. This can avoid failed testing due to improperly prepared blood samples, which can significantly delay results and thus patient care.

It is an important advantage of the present disclosure to be able to provide fast and accurate hematological analysis of a blood sample, such as through blood gas content and blood count content. Further, it is an important advantage of the present disclosure to be able to properly prepare a blood sample for certain hematological analysis. This can avoid failed testing due to improperly prepared blood samples, which can significantly delay results and thus patient care. Further, it is an important advantage to provide point-of-care hematological results, rather than having to transport the blood to a laboratory setting.

The present disclosure allows for devices which can allow for parallel hematological analysis, thus providing faster and more accurate results. Additionally, the present disclosure allows for devices having improved preparation of blood samples for hematological analysis, in particular though the use of adjustable proper dosing of the blood sample with reagent. This can further improve the final results of the analysis, such as providing an accurate complete blood cell count. Further, advantageously the preparation, and eventual analysis, can all be done at a point-of-care facility, such as an emergency room.

Further, the present disclosure provides an extensive number of blood parameters facilitating fast diagnosis and treatment of different conditions.

### BRIEF DESCRIPTION OF THE DRAWINGS

The above and other features and advantages of the present disclosure will become readily apparent to those skilled in the art by the following detailed description of example embodiments thereof with reference to the attached drawings, in which:

FIG. 1A illustrates a schematic of an example blood analyzer arranged in parallel as disclosed herein,

FIG. 1B illustrates a schematic of an example blood analyzer as disclosed herein,

FIG. 2 illustrates a schematic of an example blood count sensor as disclosed herein,

FIG. 3 illustrates a schematic of an example blood analyzer arranged in series as disclosed herein,

FIG. 4 illustrates a schematic of an example helical mixer as disclosed herein,

FIG. 5 illustrates a schematic cross-section of an example helical mixer as disclosed herein,

FIG. 6 illustrates a flow chart of example method steps as disclosed herein, and

FIGS. 7A-7B illustrate Dean flow vortices of an example helical mixer as disclosed herein.

### DETAILED DESCRIPTION

Various example embodiments and details are described hereinafter, with reference to the figures when relevant. It should be noted that the figures may or may not be drawn to scale and that elements of similar structures or functions are represented by like reference numerals throughout the figures. It should also be noted that the figures are only intended to facilitate the description of the embodiments. They are not intended as an exhaustive description of the invention or as a limitation on the scope of the invention. In addition, an illustrated embodiment needs not have all the aspects or advantages shown. An aspect or an advantage described in conjunction with a particular embodiment is not necessarily limited to that embodiment and can be practiced in any other embodiments even if not so illustrated, or if not so explicitly described.

Disclosed herein are examples of blood analyzers, e.g., devices, systems, blood sensors, blood analyzer systems, hematological analyzers, hematology analyzers, blood testers, blood sensors, blood analyzer devices. Advantageously, one or more example blood analyzers can be used to analyze multiple blood parameters. The analysis of the multiple blood parameters can be performed sequentially or in parallel. For example, the blood analyzer can incorporate one or more sensor devices. Each sensor device may analyze a different blood parameter. Each of the sensor devices can have different blood preparation requirements, if any, for a blood sample to be analyzed. Accordingly, one or more example blood analyzers may be able to prepare a blood sample prior to the blood sample being analyzed by a particular sensor device.

In one or more example blood analyzers, the blood analyzer may include an inlet module. The inlet module can be configured to receive an initial blood sample. The inlet module can be configured to receive a plurality of initial blood samples.

In one or more example blood analyzers, the blood analyzer can further include a blood gas sensor device. The blood gas sensor device can be in fluid communication with the inlet module. The blood gas sensor device can be configured to receive a first blood sample of the initial blood sample. The blood gas sensor can be configured to conduct a blood gas analysis on the first blood sample. The blood gas sensor can be configured to determine a blood gas analysis parameter.

In one or more example blood analyzers, the blood analyzer can include a blood count sensor device. The blood count sensor device can be in fluid communication with the inlet module. The blood count sensor device can be configured to receive a second blood sample of the initial blood sample. The blood count sensor device can be configured to determine a blood count parameter.

In one or more example blood analyzers, the blood analyzer can include an output. The output can be configured to provide the blood gas analysis parameter. The output can be configured to provide the blood count parameter. The output may include a display and/or a printer.

As discussed herein, the inlet module, the blood gas sensor device and/or the blood count sensor device and/or the computer system and/or the output and/or the reagent module may be considered or recited as modules, for example modules of the blood analyzer.

In one or more example blood analyzers, the blood gas sensor device and the blood count sensor device can be connected to the inlet module in parallel. In one or more example blood analyzers, the blood gas sensor device and the blood count sensor device can be connected in series. For example, the blood gas sensor device can be closer to the inlet module than the blood count sensor device.

Advantageously, one or more example blood analyzers can be used for point-of-care hematology testing (e.g., at the time and place of patient care). Thus, the blood analyzer can be operated quickly and easily, such as in a patient's room. In one or more example blood analyzers, a blood sample can be taken directly from the patient to the blood analyzer within the same room. For example, the blood analyzer can be located in a diagnostic room and/or testing room and/or hospital room and/or surgery room and/or operating room. Accordingly, blood sample can be taken from a patient and tested in the same area without transporting the blood sample. This can advantageously lead to the avoidance of transportation mix-ups or damage to the blood sample. Further, one or more example blood analyzers can provide for significantly faster hematological analysis and results, thereby leading to improved patient care. Thus, the blood sample need not be taken to a laboratory, and the preparation and analysis can be performed in a single room, such as with the patient in it. Thus, the blood analyzer can be a point of care system.

Alternatively, the disclosed blood analyzers may be used outside of point-of care hematology testing, such as laboratory hematology testing. Thus, the blood sample may be sent away from the patient to a separate location with a blood analyzer.

Generally, one or more example blood analyzers of the disclosure can be configured to properly prepare, e.g. treat, modify, pretreat, a blood sample for hematological testing, e.g., analysis, assessment. The blood sample may be taken directly from a patient. The blood sample may be from a storage. The blood sample can be from a human and/or an animal. The blood sample may be an artificial blood sample.

Advantageously, one or more example blood analyzers can provide advantages for hematological analysis. For example, the overall time until blood gas and/or blood count analysis can be greatly reduced. As discussed, the blood analyzer can provide a blood gas analysis parameter and/or a blood count parameter. In one or more example blood analyzers, the overall time until blood gas and/or blood count results are analyzed can be less than 180, 170, 160, 150, 140, 130, 120, 110, 100, 90, 80, 70, 60, 55, 50, 45, 40, 35, 30, 25, 20, 15, 10, or 5 seconds. In one or more example blood analyzers, the overall time until blood gas and/or blood count results are analyzed can be greater than 180, 170, 160, 150, 140, 130, 120, 110, 100, 90, 80, 70, 60, 55, 50, 45, 40, 35, 30, 25, 20, 15, 10, or 5 seconds. The analysis and parameters can be provided via an output. The output may provide the blood gas analysis parameter and the blood count parameter within 180 seconds, such as less than 120 seconds, after insertion of the initial blood sample into the inlet module. The output may provide the blood gas analysis parameter and the blood count parameter within 30 seconds.

Moreover, one or more example blood analyzers can allow for improved hematological analysis, such as blood count analysis, such as white blood cell and/or platelet count analysis. For example, the blood analyzer is configured to achieve full blood cell lysis and strong white blood cell and/or platelet staining for the blood count sensor device.

Full, e.g. sufficient, blood cell lysis can be achieved when 95, 96, 97, 98, 99, 99.5, 99.6, 99.7, 99.8, 99.9, or 100% of red blood cells are lysed. Full, e.g. sufficient, blood cell lysis can be achieved when less than 95, 96, 97, 98, 99, 99.5, 99.6, 99.7, 99.8, 99.9, or 100% of red blood cells are lysed. Full, e.g. sufficient, blood cell lysis can be achieved when greater than 95, 96, 97, 98, 99, 99.5, 99.6, 99.7, 99.8, or 99.9% of red blood cells are lysed.

Advantageously, one or more of example blood analyzers can achieve minimal white blood cell and/or platelet lysing. In one or more example blood analyzers, 5, 4, 3, 2, 1, 0.5, 0.4, 0.3, 0.2, 0.1, or 0.0% of white blood cells and/or platelets are lysed. In one or more example blood analyzers, less than 5, 4, 3, 2, 1, 0.5, 0.4, 0.3, 0.2, or 0.1% of white blood cells and/or platelets are lysed. In one or more example blood analyzers, greater than 5, 4, 3, 2, 1, 0.5, 0.4, 0.3, 0.2, 0.1, or 0.0% of white blood cells and/or platelets are lysed.

Strong white blood cell and/or platelet staining can be achieved when 95, 96, 97, 98, 99, 99.5, 99.6, 99.7, 99.8, 99.9, or 100% of white blood cells and/or platelets are stained. Strong white blood cell and/or platelet staining can be achieved when less than 95, 96, 97, 98, 99, 99.5, 99.6, 99.7, 99.8, 99.9, or 100% of white blood cells and/or platelets are stained. Strong white blood cell and/or platelet staining can be achieved when greater than 95, 96, 97, 98, 99, 99.5, 99.6, 99.7, 99.8, or 99.9% of white blood cells and/or platelets are stained.

Thus, one or more example blood analyzers allows for the integration, or semi-integration, of a blood count sensor device with a blood gas sensor device. The devices can include shared fluid pathways and can analyze one initial blood sample. This can greatly speed up the analysis process, and allow for cooperation between the devices to achieve a highly accurate result. As typically a user would require two different machines, one or more example blood analyzers can save space in a room, especial for POC, and would not require multiple blood draws. Thus a single blood draw can be used by both integrated devices.

The blood analyzer may include and/or be associated with a housing, such as a single housing. The housing can accommodate any and/or all of the modules discussed herein. For example, the housing can accommodate an inlet module, a blood gas sensor device, a blood count sensor device, and an output. In one or more example blood analyzers, the housing can accommodate an inlet module, a blood gas sensor device, and a blood count sensor device, whereas the results of the parameter analysis can be output to a different device, such as a laptop, or to a remote system. In one or more example blood analyzers, one or more of the modules of the blood analyzer discussed herein can include a separate module housing within the housing. For example, the blood gas sensor device may include and/or be located and/or associated with a module housing within the housing. For example, the blood count sensor device may include and/or be located and/or associated with a module housing within the housing.

In one or more example blood analyzers, the housing can further include a computer system. In one or more example blood analyzers, the housing can be configured to communicate with a computer system outside of the housing. In one or more example blood analyzers, the housing can include actuators, e.g., switches, buttons, knobs, keyboards, touch screens, for operating the analyzer and/or any specific modules of the analyzer.

Alternatively, in one or more example blood analyzers, the blood count sensor device and the blood gas sensor device may be in separate housings, such as a first housing and a second housing. Thus, the devices may be separated, e.g. physically separated. The blood count sensor device and blood gas sensor device may each be fluidly connected to an inlet module. The inlet module may be on the first housing, the second housing, or a third housing.

The housing may be plastic, metal, ceramic, etc. or combinations thereof, and the particular material of the housing is not limiting. The housing may include one or more ports. The housing may include one or more slots. The housing may include one or more vents. The housing may include electronics, such as wiring for data and/or wiring for power. The housing may be a frame for holding the different modules of the blood analyzers.

In one or more example blood analyzers, the blood analyzer can include an inlet module (e.g., inlet, opening, slot, receiver, aperture, gap). The inlet module may be located on and/or associated with the housing. The inlet module can be configured to receive an initial blood sample. The inlet module may provide a fluid connection between externals of the blood analyzer and internals of the blood analyzer. The initial blood sample can be taken from a patient.

The initial blood sample can be received in a container. The container could be a tube. The container could be a capillary tube. The container could be a cylinder. The container could be a cuvette. The container can be a syringe. The container could be a vacuum tube.

The container can be sized and configured to fit within an inlet module in the blood analyzer. The container can be sized and configured to fit within an inlet (e.g., inlet module, opening, aperture, gap, slot, receiver) in the inlet module for providing the initial blood sample into the blood analyzer. The container may provide or enable a fluid connection between an initial blood sample and the inlet of the inlet module. The container can be a single-use container. The particular type of inlet module is not limiting to the disclosure.

In one or more example blood analyzers, the inlet module may be further configured to aspirate the initial blood sample from the container. In one or more example blood analyzers, the inlet module may not be configured to aspirate the initial blood sample.

The inlet module can be configured to receive a single initial blood sample. Thus, the single initial blood sample can be received in a complete integration between the blood gas sensor device and the blood count sensor device. The inlet module can be configured to receive more than one initial blood sample. Thus, the more than one initial blood sample can be received in a semi-integration between the blood gas sensor device and the blood count sensor device. For example, the inlet module can receive a first initial blood sample and a second initial blood sample at the same time. Thus, the inlet module may contain more than one inlet. In alternate blood analyzers, the inlet module can receive a first initial blood sample into an inlet first, followed by a second initial blood sample into the same inlet while the first initial blood sample is being analyzed.

In one or more example blood analyzers, the inlet module can be configured to receive cleaning fluid, e.g. cleaner, sterilizer, cleaning solution, cleaning liquid, such as from a cleaning module. The blood analyzer, e.g. the inlet module, may include a cleaning module. The cleaning module may be separate from the inlet module. The cleaning liquid can pass through the blood analyzer and clean the various modules and/or fluid pathway(s) discussed herein. The inlet module can receive the cleaning fluid in the same manner as the initial blood sample. Alternatively, the inlet module can include an additional inlet for receiving the cleaning fluid from the cleaning module.

The inlet module can be configured to receive a quality control (QC) bead solution. The inlet module can be configured to receive a dye solution. The inlet module may have an inlet configured to refill the reagent reservoir discussed herein.

In one or more example blood analyzers, the inlet module may include 1, 2, 3, 4, 5 or 6 inlets. In one or more example blood analyzers, the inlet module may include greater than 1, 2, 3, 4, 5 or 6 inlets. In one or more example blood analyzers, the inlet module may include less than 1, 2, 3, 4, 5 or 6 inlets.

The inlet module may be configured to receive an initial blood sample having a volume of 5, 10, 15, 20, 25, 30, 35, 40, 45, 50, 60, 70, 80, 90, 100, 110, 120, 130, 140, 150, 160, 170, 180, 190, or 200 μL. The inlet module may be configured to receive an initial blood sample having a volume of greater than 5, 10, 15, 20, 25, 30, 35, 40, 45, 50, 60, 70, 80, 90, 100, 110, 120, 130, 140, 150, 160, 170, 180, 190, or 200 μL. The inlet module may be configured to receive an initial blood sample having a volume of less than 5, 10, 15, 20, 25, 30, 35, 40, 45, 50, 60, 70, 80, 90, 100, 110, 120, 130, 140, 150, 160, 170, 180, 190, or 200 μL. In one or more example blood analyzers, the initial blood sample is less than 150 μL, such as less than 100 μL.

In one or more example blood analyzers, the initial blood sample may be mixed (e.g., pretreated) with an anticoagulant, e.g., blood thinner. Thus the initial blood sample may include an anticoagulant. The anticoagulant may include, but is not limited to ethylenediaminetetraacetic acid (EDTA), coumarins, heparins, synthetic pentasaccharides, citrate, Iloprost, beraprost, MgSO₄ tubes, and their derivatives/alternatives and combinations. The anticoagulant may be added into the initial blood sample prior to entering the blood analyzer. In one or more example blood analyzers, the anticoagulant can be in a container receiving the initial blood sample from the patient. In alternative analyzers, the initial blood sample is not mixed with an anticoagulant. The inclusion of the anticoagulant can be part of the preparation. The anticoagulant may be part of the initial blood sample itself. In one or more example blood analyzers, the inlet module may be configured to provide an anticoagulant into the initial blood sample.

The inlet module may be configured to receive an initial blood sample with coagulant having a total volume of 5, 10, 15, 20, 25, 30, 35, 40, 45, 50, 60, 70, 80, 90, 100, 110, 120, 130, 140, 150, 160, 170, 180, 190, or 200 μL. The inlet module may be configured to receive an initial blood sample with coagulant having a total volume of greater than 5, 10, 15, 20, 25, 30, 35, 40, 45, 50, 60, 70, 80, 90, 100, 110, 120, 130, 140, 150, 160, 170, 180, 190, or 200 μL. The inlet module may be configured to receive an initial blood sample with coagulant having a total volume of less than 5, 10, 15, 20, 25, 30, 35, 40, 45, 50, 60, 70, 80, 90, 100, 110, 120, 130, 140, 150, 160, 170, 180, 190, or 200 μL.

Advantageously, if anticoagulant is used, the same anticoagulant can be used in the blood count sensor device and the blood gas sensor device without affecting the results of the two devices. Thus, a single anticoagulant can be used that can be used for both devices, and the blood sample with the anticoagulant can be accurately analyzed by both devices. For example, Iloprost and/or heparin and their derivatives/alternatives and combinations can be used in both the blood gas sensor device and the blood count sensor device. On the other hand, EDTA may not be used for a blood gas sensor device.

In one or more example blood analyzers, saline can be added into the initial blood sample. This can dilute the sample. The dilution may be taken into consideration for one or more of the analyzers detailed below. In alternative embodiments, no saline is added to the initial blood sample. Thus, the initial blood sample may not be diluted with saline.

Once the initial blood sample is received from the inlet module, it may follow one or more fluid pathways in the blood analyzer, such as in the housing. Thus, the inlet module is fluidly connected with the fluid pathway(s). These fluid pathways can be, for example, tubes, silicon tubes, fluorinated ethylene propylene (FEP) tubes, pipes, capillaries, conduits, hoses, ducts, etc. The fluid pathway may be flexible. The fluid pathway may be rigid. The fluid pathway may be flexible and include rigid components. The fluid pathway may be rigid and include flexible components.

The fluid pathway may have the same diameter throughout the blood analyzer. The fluid pathway may have different diameters through the blood analyzer.

One or more of the inlets of the inlet module may lead to a single initial fluid pathway. Thus, the modules discussed herein can be fluidly connected in series. The initial blood sample can pass through the different modules one after one another. For example, the initial blood sample can pass through the inlet module, followed by the blood gas sensor device, followed by the blood count sensor device. Alternatively, the initial blood sample can pass through the inlet module, followed by the blood count sensor device, followed by the blood gas sensor device. The inlet module, blood gas sensor device, and blood count sensor device can be fluidly connected.

One or more of the inlets may lead to a separate fluid pathway. For example, the inlet module can include a first inlet leading to a first fluid pathway and a second inlet leading to a second fluid pathway, where the first fluid pathway and the second fluid pathway are not in fluid communication. Of the initial blood sample, the first inlet may accept a first blood sample and the second inlet may accept a second blood sample. The order of acceptance does not matter, and a user can start with the first inlet or the second inlet. Each inlet can be configured to receive a particular volume of blood and shut off. Thus, starting with the initial blood sample, a user can insert the initial blood sample into the first inlet to draw the first blood sample, and then can take the remainder to the second inlet.

Thus, in one or more example blood analyzers, the inlet module can include a divider. The divider can be configured to divide the initial blood sample. For example, the diver can divide the initial blood sample into a first blood sample and a second blood sample. The divider can divide the initial blood sample into two particular volumes. Thus, a single initial blood sample can be inserted into the inlet module into an initial fluid pathway, and the divider can divide the initial blood sample into the first blood sample and the second blood sample. The divider can be a rotary valve. The divider can be a valve. The divider can be a dosing valve.

In one or more example blood analyzers, the initial blood sample can be inserted into the inlet module, such as in a container. The inlet module may allow the first blood sample to pass from the initial blood sample into the initial fluid pathway and then the first fluid pathway. The inlet module then may allow the second blood sample to pass from the initial blood sample into the initial fluid pathway and then the second fluid pathway.

Thus, the inlet module may include devices, e.g. systems, mechanisms, to take a first blood sample and a second blood sample from the initial blood sample.

Accordingly, the inlet module may lead to an initial fluid pathway, and the initial fluid pathway may branch into a first fluid pathway and a second fluid pathway. The split may be a fluid pathway branch. The first fluid pathway may lead to the blood gas sensor device. The second fluid pathway may lead to the blood count sensor device.

Advantageously, splitting the initial blood sample into the first blood sample for the first fluid pathway and the second blood sample for the second fluid pathway may allow for parallel analysis of the initial blood sample. The parallel analysis can occur simultaneously. For example, the blood gas sensor device can analyze the first blood sample to determine the blood gas analysis parameter while the blood count sensor device can analyze the second blood sample to determine the blood count analysis parameter.

Besides time optimization, the parallel analysis can decouple the two measurements and allow rinsing of the blood gas sensor device while the blood count sensor device still measures. Otherwise, the blood gas sensor device would show an unacceptable drift because of a long exposure to the sample.

As mentioned, the initial blood sample may be divided into the first blood sample and the second blood sample. The first blood sample and the second blood sample may have equal volumes. The first blood sample may have a volume greater than the second blood sample. The first blood sample may have a volume less than the second blood sample.

In one or more example blood analyzers, the first blood sample may proceed to a blood gas sensor device.

The first blood sample may be 5, 10, 15, 20, 25, 30, 35, 40, 45, 50, 60, 70, 80, 90, or 100 μL. The first blood sample may be greater than 5, 10, 15, 20, 25, 30, 35, 40, 45, 50, 60, 70, 80, 90, or 100 μL. The first blood sample may be less than 5, 10, 15, 20, 25, 30, 35, 40, 45, 50, 60, 70, 80, 90, or 100 μL. In one or more example blood analyzers, the first blood sample can have a volume less than 100 μL, such as less than 80 μL, or less than 50 μL.

In one or more example blood analyzers, the second blood sample can proceed to the blood count sensor device.

The second blood sample may be 5, 10, 15, 20, 25, 30, 35, 40, 45, or 50 μL. The second blood sample may be greater than 5, 10, 15, 20, 25, 30, 35, 40, 45, or 50 μL. The second blood sample may be less than 5, 10, 15, 20, 25, 30, 35, 40, 45, or 50 μL. In one or more example blood analyzers, the second blood sample has a volume less than 50 μL, such as less than 30 μL, in the range from 10 μL to 25 μL.

In one or more example blood analyzers, following the initial blood sample from the inlet module, the blood analyzer can include a first sensor, e.g. first sensor module, detector, electronic, associated with the fluid pathway, such as the initial fluid pathway and/or the first fluid pathway and/or the second fluid pathway. The first sensor may be associated with the inlet module. The first sensor can be one or more sensors. The first sensor may be along the fluid pathway, such as the initial fluid pathway and/or the first fluid pathway and/or the second fluid pathway, in order to obtain sensor data while the initial blood sample and/or the first blood sample and/or the second blood sample passes through the first sensor.

In one or more example blood analyzers, the first sensor may be located before the fluid pathway branch. In one or more example blood analyzers, the first sensor may be located after the fluid pathway branch. In particular, the first sensor may be located in the second fluid pathway. In one or more example blood analyzers, the first sensor may be located in the fluid pathway branch.

The first sensor may be configured to determine one or more blood parameters, such as a first blood parameter as discussed below. The first sensor may output data indicative of one or more blood parameters. The first sensor may also be configured to capture an image and provide image data. The terms image data and sensor data may be used interchangeably herein. The first sensor may output the sensor data. In one or more example blood analyzers, the blood analyzer may not include a first sensor.

The first sensor may be an image capturer. The first sensor may be a camera. The first sensor may be an imaging system. The first sensor may be a turbidity sensor. The first sensor may be a liquid sensor. The first sensor may be a modified liquid sensor. The first sensor may be a porous mirror. The first sensor may be a red blood cell parameter sensor. The first sensor may be a spectrophotometer. The first sensor may be an electro/impedance measuring unit. The first sensor may be an optical diffraction tomography unit. The first sensor may be a transmittance sensor. The first sensor may be a refractive index sensor. The first sensor may be a hematocrit (Hct) sensor. The Hct sensor may be used to adapt dosing volume, such as for low Hct samples.

The first sensor may be a hemolysis detector. The hemolysis detector can be sensitive to refraction index changes from lipids. Thus, the hemolysis detector can detect lipid levels. As mentioned, the first sensor may be an impedance sensor. The impedance sensor may provide sensor data, which may be indicative of the first blood parameter. The impedance sensor may provide a first blood parameter indicative of electrolyte content. The impedance sensor may provide a first blood parameter indicative of lipid content. The impedance sensor may provide a first blood parameter indicative of hematocrit content. The impedance sensor may provide a first blood parameter indicative of electrolyte content and/or lipid content and/or hematocrit content.

The first sensor may be configured to sense one or more electrical and/or optical properties.

In one or more example blood analyzers, the first sensor may be configured for performing a conductivity analysis. The first sensor can determine the hematocrit, e.g. the volume percentage of red blood cells. For example, electrodes can be used to measure the conductivity. Osmolality can be measured/estimated from the conductivity. The electrodes can span the fluid pathway in order to obtain sensor data. The electrodes can be incorporated into the fluid pathway.

The first sensor may be an absorbance sensor. Thus, the first sensor can perform a light intensity analysis e.g. at one or more different wavelengths. For example, an optical sensor can be used. The optical sensor and/or light source can be at 509, 522, 549, 569, 587, 650, 660, 670, 680, or 690 nm.

In one or more example blood analyzers, the first sensor can be configured to perform a viscosity analysis of the initial blood sample.

In one or more example blood analyzers, the first sensor can be configured to perform a pH analysis of the initial blood sample.

In one or more example blood analyzers, the first sensor can be configured to perform a hematocrit analysis.

In one or more example blood analyzers, the first sensor may obtain the first blood parameter directly. In one or more example blood analyzers, the first sensor may obtain the first blood parameter indirectly. For example, the first sensor may measure a sum of electrolytes and similar analytes of the osmolality, which can affect the ability to lyse and stain cells.

In one or more example blood analyzers, multiple sensors and/or image capturers may be used. Thus, multiple different parameters can be obtained from the initial blood sample. Alternatively, multiple sensors and/or image capturers may be used to obtain and verify the same blood parameter.

As mentioned, the first sensor may be configured to monitor the fluid pathway, such as the initial fluid pathway and/or the first fluid pathway and/or the second fluid pathway, and provide sensor data. The sensor data may include, directly or indirectly, a first blood parameter. The first blood parameter may be any number of parameters of the initial blood sample and/or the first blood sample and/or the second blood sample. In one or more example blood analyzers, the first blood parameter may be a red blood cell content. In one or more example blood analyzers, the first blood parameter may be a plasma content. In one or more example blood analyzers, the first blood parameter may be a lipid content. For example, the lipid content may be from a sample quality index called hemolysis, icterus, lipemia (HIL) index. In one or more example blood analyzers, the first blood parameter may be a white blood cell content. In one or more example blood analyzers, the first blood parameter may be a blood gas content. In one or more example blood analyzers, the first blood parameter may be a platelet content. In one or more example blood analyzers, the first blood parameter may be a protein content. In one or more example blood analyzers, the first blood parameter may be a cholesterol content. In one or more example blood analyzers, the first blood parameter may be total cholesterol content. In one or more example blood analyzers, the first blood parameter may be low-density lipoprotein (LDL) content. In one or more example blood analyzers, the first blood parameter may be high density lipoprotein (HDL) content. In one or more example blood analyzers, the first blood parameter may be triglyceride content. In one or more example blood analyzers, the first blood parameter may be a hematocrit content. In one or more example blood analyzers, the first blood parameter may be glucose content. In one or more example blood analyzers, the first blood parameter may be electrolyte content. The first blood parameter may be more than one of the parameters discussed herein, e.g. a combination of two or more. Other first blood parameters can be used as well, and the particular blood parameter is not limiting.

Thus, the first sensor may be configured to detected and/or analyze and/or determine and/or provide any of the above first blood parameters. The first sensor may be configured to detect a plurality of the above first blood parameters.

The first sensor may be configured to provide and/or output sensor data, such as the first blood parameter. For example, the first sensor may output the sensor data to a computer system, for example a local and/or a remote computer system, as discussed in detail below. The first sensor may output the sensor data to a visual device for review by a user.

As mentioned, the initial fluid pathway and/or first fluid pathway may lead to a blood gas sensor device, e.g. analyzer, system, housing, machine, module. The blood gas sensor device may be configured to determine a blood gas analysis parameter. The blood gas sensor device may be configured to provide the blood gas analysis parameter to the computer system, such as discussed below. The blood gas sensor device may be configured to provide the blood gas analysis parameter to the blood count sensor device. Thus, the blood gas analysis device may be configured to provide a blood gas (BG) analysis. The blood gas sensor device may be an Acid Base Laboratory (ABL) analyzer.

The blood gas analysis device may utilize wet chemistry. The blood gas analysis device may not utilize wet chemistry. The blood gas analysis device may utilize dry chemistry. The blood gas analysis device may not utilize dry chemistry. The blood gas analysis device may not utilize any further chemistry.

In one or more example blood analyzers, the blood gas analysis parameter may be the first blood parameter discussed above. Thus, the first sensor may not be used, and the first blood parameter can be determined by the blood gas sensor device. In one or more example blood analyzers, the blood gas analysis parameter may be different from the first blood parameter.

The blood gas sensor device may include an oximetry sensor. The blood gas sensor device may include a blood metabolic panel (BMP) plate. The blood gas sensor device may include a hemolysis detector. The blood gas sensor device may include an electrolyte sensor. The blood gas sensor device may include any or all of the above-listed components.

The blood gas analysis parameter may be blood gas level. The blood gas analysis parameter may be oxygen saturation. blood gas analysis parameter may be SpO₂. The blood gas analysis parameter may be pH. The blood gas analysis parameter may be pCO₂ concentration. The blood gas analysis parameter may be pO₂ concentration. The blood gas analysis parameter may be electrolyte concentration, such as cCa2⁺, cCl⁻, cK⁺, cNa⁺. The blood gas analysis parameter may be metabolite concentration, such as cGlu or cLac. The blood gas analysis parameter may be oximetry (CO—OX), such as providing COHb, ctBil, ctHb, FHbF, FHHb, MetHb, sO₂, and FO₂Hb. The blood gas analysis parameter may be cCrea and/or cUrea/BUN.

In one or more example blood analyzers, the blood gas sensor device can provide the blood gas analysis parameter in less than 60, 55, 50, 45, 40, 35, 30, 25, 20, 15, 10, or 5 seconds from receiving the initial blood sample in the inlet module. In one or more example blood analyzers, the blood gas sensor device can provide the blood gas analysis parameter in greater than 60, 55, 50, 45, 40, 35, 30, 25, 20, 15, 10, or 5 seconds from receiving the initial blood sample in the inlet module.

In one or more example blood analyzers, the analyzer can include a reagent module, e.g. system, device, mechanism. The reagent module can be configured to provide one or more reagents to a blood sample, such as the initial blood sample and/or the second blood sample. The reagent module may be part of the blood count sensor device, discussed below. The reagent module may be separate from the blood count sensor device.

In one or more example blood analyzers, the reagent module can be in fluid connection with the second fluid pathway. For example, this can be used for the parallel analysis. In one or more example blood analyzers, the reagent module can be in fluid connection with a single fluid pathway, such as the initial fluid pathway. For example, this can be used for the series analysis.

In one or more example blood analyzers, the reagent module is separated from the blood gas sensor device. Specifically, the reagents provided by the reagent module may adversely affect the blood gas sensor device and the resulting blood gas analysis parameter. Thus, the reagent module may be located separate from and/or after the blood gas sensor device in order to not interfere with the blood gas sensor device.

The reagent module may include a reagent reservoir. The reagent reservoir can be configured to accommodate a reagent. In one or more example blood analyzers, the reagent module may include a first reagent reservoir and a second reagent reservoir. The first reagent reservoir can be configured to accommodate a first reagent and the second reagent reservoir can be configured to accommodate a second reagent. The reagent module may include a third, fourth, etc. reservoir to accommodate a respective third, fourth, etc. reagent.

The reagent reservoir may hold a designated consumable. The reagent reservoir may be refilled. For example, the reagent reservoir may be filled with a solution pack. The solution pack may be shared between the blood gas sensor device and the blood count sensor device.

The reagent reservoir may be filled with solutions other than reagent. For example, the reagent reservoir may hold QC bead solution. The reagent reservoir may hold cuvette calibration dye. Alternatively, the QC bead solution and/or the cuvette calibration dye may be located in separate a separate solution reservoir and/or a separate dye reservoir.

The reagent module may further include a reagent inlet, e.g., injection point, injection port, injector. The reagent inlet may be in fluid connection with the reagent reservoir and the fluid pathway, such as the initial fluid pathway and/or the second fluid pathway. The reagent module may include a second, third, etc. reagent inlet in fluid connection with the second, third, etc. reagent reservoir.

The reagent reservoir and/or the reagent inlet may be a syringe pump.

The reagent inlet may include a dosing meter, e.g. dosing system, dosing device, dosing mechanism. The dosing meter may be configured to provide a particular dose, e.g. volume, size, amount, of reagent into the fluid pathway, such as the initial fluid pathway and/or the second fluid pathway. The dosing meter may be adjusted, so a first volume of reagent can be provided, and a second volume of reagent can be provided. The first volume may be different from the second volume.

The reagent module may include a reagent module sensor. The reagent module sensor may be a liquid sensor. The reagent module sensor may trigger dosing of the reagent. For example, the reagent module sensor may trigger once the blood sample, such as the initial blood sample and/or the second blood sample, passes the module sensor.

The reagent module may be configured to prepare a blood sample, such as the initial blood sample and/or the second blood sample, for analysis by the blood count sensor. In particular, the reagent module can be configured to provide one or more of a volume of reagent to the blood sample, such as the initial blood sample and/or the second blood sample.

In one or more example blood analyzers, the reagent module may be configured to provide an initial volume of a reagent. The initial volume of the reagent can be in the range from 0.5 to 20 times a volume of the blood sample, such as the initial blood sample and/or the second blood sample. The initial volume can be 0.5, 1, 2, 3, 4, 5, 6, 7, 8, 9, 10, 11, 12, 13, 14, 15, 16, 17, 18, 19, or 20 times the volume of the blood sample, such as the initial blood sample and/or the second blood sample. The initial volume can be less than 0.5, 1, 2, 3, 4, 5, 6, 7, 8, 9, 10, 11, 12, 13, 14, 15, 16, 17, 18, 19, or 20 times the volume of the blood sample, such as the initial blood sample and/or the second blood sample. The initial volume can be greater than 0.5, 1, 2, 3, 4, 5, 6, 7, 8, 9, 10, 11, 12, 13, 14, 15, 16, 17, 18, 19, or 20 times the volume of the blood sample, such as the initial blood sample and/or the second blood sample.

In one or more example blood analyzers, a blood sample, such as the initial blood sample and/or the second blood sample, to reagent ratio can be 1:1, 1:2, 1:3, 1:4, or 1:5. Accordingly, 20 μL of a blood sample, such as the initial blood sample and/or the second blood sample, can have 20, 30, 40, 50, 60, 70, 80, 90, or 100 μL of reagent added. In one or more example blood analyzers, the blood sample, such as the initial blood sample and/or the second blood sample, to reagent ratio can be greater than 1:1, 1:2, 1:3, 1:4, or 1:5. In one or more example blood analyzers, the blood sample, such as the initial blood sample and/or the second blood sample, to reagent ratio can be less than 1:1, 1:2, 1:3, 1:4, or 1:5. In particular, the ratio of blood sample, such as the initial blood sample and/or the second blood sample, to reagent can be significantly below 1:10000, 1:200, 1:100, and 1:50 as typically required in laboratory settings.

The initial volume of the reagent may be 5, 10, 15, 20, 25, 30, 35, 40, 45, 50, 60, 70, 80, 90, 100, 110, or 120 μL. The initial volume of the reagent may be greater than 5, 10, 15, 20, 25, 30, 35, 40, 45, 50, 60, 70, 80, 90, 100, 110, or 120 μL. The initial volume of the reagent may be less than 5, 10, 15, 20, 25, 30, 35, 40, 45, 50, 60, 70, 80, 90, 100, 110, or 120 μL.

The reagent can be one or more reagents that can be mixed with, e.g. incorporated into, combined with, added into, the blood sample, such as the initial blood sample and/or the second blood sample. The reagent can be configured to affect one or more properties of the blood sample, such as the initial blood sample and/or the second blood sample. For example, the reagent can be a staining reagent, e.g. stain the blood sample, such as the initial blood sample and/or the second blood sample. This can make certain components of the blood, such as white blood cells and platelets, optically visible for analysis. The reagent can be a lysing reagent, e.g. to lyse the blood sample, such as the initial blood sample and/or the second blood sample. The lysing can affect the red blood cells. A single reagent can be a lysing reagent and a staining reagent. Multiple reagents could be used as well, with one reagent being a lysing reagent and another being a staining reagent.

Thus, the reagent can be configured to lyse the blood sample, such as the initial blood sample and/or the second blood sample. The reagent can be configured to stain the blood sample, such as the initial blood sample and/or the second blood sample. The reagent is configured to lyse and stain the blood sample, such as the initial blood sample and/or the second blood sample. The reagent is configured to lyse and/or stain the blood sample, such as the initial blood sample and/or the second blood sample.

In one or more example blood analyzers, the reagent module can be configured to provide a first volume of a second reagent, e.g. configured to adjust reagent levels. The second reagent may be accommodated by the second reservoir. A second reagent may be used for a number of reasons. For example, the first reagent may be a lysing reagent, and the second reagent may be a staining reagent. Alternatively, the first reagent may be a staining reagent, and the second reagent may be a lysing reagent. In one or more example blood analyzers, the first reagent can be a staining and/or lysing reagent. In one or more example blood analyzers, the second reagent is a staining and/or lysing reagent.

In one or more example blood analyzers, the first reagent may be a lysing and staining reagent, and the second reagent may be a lysing and staining reagent. The second reagent may be a weaker lysing and staining reagent than the first reagent. The second reagent may be a stronger lysing and staining reagent than the first reagent.

The second reagent may have a stronger lysing component and the same staining component as the first reagent. The second reagent may have a weaker lysing component and the same staining component as the first reagent. The second reagent may have a stronger lysing component and a weaker staining component as the first reagent. The second reagent may have a weaker lysing component and a stronger staining component as the first reagent.

The first volume of second reagent may be greater than the initial volume of first reagent. The first volume of second reagent may be less than the initial volume of first reagent. The first volume of second reagent may be the same as the initial volume of first reagent.

The first volume of second reagent may be greater than the first volume of first reagent. The first volume of second reagent may be less than the first volume of first reagent. The first volume of second reagent may be the same as the first volume of first reagent.

The first volume of the second reagent may be 5, 10, 15, 20, 25, 30, 35, 40, 45, 50, 60, 70, 80, 90, 100, 110, or 120 μL. The first volume of the second reagent may be greater than 5, 10, 15, 20, 25, 30, 35, 40, 45, 50, 60, 70, 80, 90, 100, 110, or 120 μL. The first volume of the second reagent may be less than 5, 10, 15, 20, 25, 30, 35, 40, 45, 50, 60, 70, 80, 90, 100, 110, or 120 μL.

Following the reagent module can be a blood count sensor device, e.g. system, housing, machine, module. The blood count sensor device may be a hematological device, such as for providing hematological analysis. The blood count sensor device may use wet chemistry. The blood count sensor device may use dry chemistry.

The blood count sensor device may be in fluid connection with the fluid pathway, such as the initial fluid pathway and/or the second fluid pathway.

In one or more example blood analyzers, the analyzer can be configured to determine and/or provide a blood count analysis parameter, such as indicative of a complete blood count (CBC) analysis. The CBC analysis can be the blood count parameter, or the blood count parameter can be indicative of the CBC analysis. In one or more example blood analyzers, the analyzer can be configured to determine and/or provide a blood sample for white blood cell differential, or white blood cell count, (WBC DIFF), e.g. including 3-part or 5-part white blood cell differential, or white blood cell count, (WBC DIFF). In one or more example blood analyzers, the analyzer can be configured to determine and/or provide a complete-blood-counting (CBC), e.g. including 3-part or 5-part CBC. As discussed herein, CBC may include a WBC DIFF, or the terms may be used interchangeably. Other components of CBC analysis may be used as well. Thus, the blood count parameter may be indicative of white blood cell content. The blood count parameter may be indicative of platelet content. The blood count sensor may be configured to provide a hematology panel.

The blood count sensor device may include one or more components. For example, the blood count sensor device can include the reagent module, such as discussed above. The reagent module can be configured to add a reagent to the second blood sample. Further, the blood count sensor can include a mixer. The mixer may be a helical mixer as disclosed herein. The mixer can be configured to mix the reagent with a blood sample, such as the initial blood sample and/or the second blood sample, thereby forming a mixture. The blood count sensor can include an incubator. The incubator can be configured to incubate the mixture and/or the blood sample and/or the reagent. The incubator can be separate from the mixer. The incubator can be combined with the mixer, such as to form a mixer-incubator. The blood analyzer can be configured so that a blood count parameter is determined from the mixture. The blood count sensor can include a cooling unit. The cooling unit can be configured to cool the mixture and/or the blood sample and/or the reagent.

The blood count sensor device can perform the analysis via artificial intelligence and/or image analysis. The analysis can be performed via a computer system, such as discussed below. The analysis can be performed via counting of the white blood cells and/or platelets.

In one or more example blood analyzers, the blood count sensor device can include a mixer. The mixer may be in fluid communication with or form a part of the fluid pathway, such as the initial fluid pathway and/or the second fluid pathway. The mixer can be located after the reagent module. Thus, the initial blood sample and/or the second blood sample and the reagent can pass through the mixer to mix the reagent with the initial blood sample and/or the second blood sample and, thereby forming a mixture.

The mixer can be a helical mixer. For example, the mixer can use a helix principle. The mixer can be a centrifuging mixer and/or a static mixer and/or a high shear mixer and/or a drum mixer. Preferably, the mixer does not activate any components of the initial blood sample, such as causing consolidation of platelets and thereby causing clotting. The mixer can provide a shear force onto the initial mixture. Increasing certain settings of the mixer, such as discussed below, can increase the likelihood of red blood cell lysis, thus providing for improved hematological analysis. For example, by increasing mixing cycles or mixing speeds, shear forces on the cells are increased, thereby improving red blood cell lysis.

The mixer can be a Dean flow mixer. The mixer can be a screw mixer. The mixer can be an involute shaped mixer. The mixer can be a diffusion mixer.

By passing through the mixer, the mixer may mix the reagent with the initial blood sample and/or the second blood sample and any subsequent mixtures. The initial blood sample and/or the second blood sample with reagent and may pass through the mixer one or more times, such as back and forth. For example, the initial blood sample and/or the second blood sample and may pass through the mixer in a first direction, thereby forming the mixture. The mixture then may reverse to a second direction and pass through the mixer again, thereby further mixing the mixture. The mixture may then reverse to the first direction to pass through the mixer a third time. Each pass through the mixer may be known as a cycle.

The mixer may include one or more mixing settings. A mixing setting can include one or more parameters of a mixer.

For example, a mixing setting can include number of mixing cycles, e.g. times that the mixture/reagent and initial blood sample and/or the second blood sample pass through the mixer, such as back and forth through at least a part of the mixer, such as the helical mixer. In one or more example blood analyzers, the mixing setting can be 1, 2, 3, 4, 5, 6, 7, 8, 9, or 10 cycles. In one or more example blood analyzers, the mixing setting can be less than 2, 3, 4, 5, 6, 7, 8, 9, or 10 cycles. In one or more example blood analyzers, the mixing setting can be greater than 1, 2, 3, 4, 5, 6, 7, 8, 9, or 10 cycles.

A mixing setting can include a flow velocity, such as a flow speed, of the mixture/reagent and the initial blood sample and/or the second blood sample through the mixer. In one or more example blood analyzers, the flow velocity can be 5, 10, 15, 20, 25, 30, 35, 40, 45, 50, 55, 60, 65, 70, 75, 80, 85, 90, 95, or 100 μL/second. In one or more example blood analyzers, the flow velocity can be greater than 5, 10, 15, 20, 25, 30, 35, 40, 45, 50, 55, 60, 65, 70, 75, 80, 85, 90, 95, or 100 μL/second. In one or more example blood analyzers, the flow velocity can be less than 5, 10, 15, 20, 25, 30, 35, 40, 45, 50, 55, 60, 65, 70, 75, 80, 85, 90, 95, or 100 μL/second.

A mixing setting can include a mixing time in the mixer. In one or more example blood analyzers, the mixing time can be 5, 10, 15, 20, 25, 30, 35, or 50 seconds. In one or more example blood analyzers, the mixing time can be greater than 5, 10, 15, 20, 25, 30, 35, or 50 seconds. In one or more example blood analyzers, the mixing time can be less than 5, 10, 15, 20, 25, 30, 35, or 50 seconds.

In one or more example blood analyzers, a helical mixer is disclosed. The helical mixer can be separate from the blood analyzer. The helical mixer can be included in the blood analyzer disclosed herein, including one or more components of the blood analyzer disclosed. The helical mixer can be a helical blood mixer. The helical mixer can be a helical fluid mixer. The helical mixer can be a helical liquid mixer. The helical mixer may include one or more helical, such as helix, components, such as discussed herein.

Disclosed herein is a helical mixer for a blood analyzer. The helical mixer can include an inlet. The inlet can be configured to receive a blood sample. The inlet can be configured to receive a reagent. The inlet can be configured to receive a blood sample and a reagent. The helical mixer can include an outlet. The outlet can be configured to output a mixture of the blood sample and the reagent. The helical mixer can include a helical flow path. The helical flow path can be extending between the inlet and the outlet. The helical mixer can be configured to mix the blood sample with the reagent.

In one or more example helical mixers, the helical mixer is for a blood analyzer, the helical mixer comprising an inlet configured to receive a blood sample and a reagent; an outlet configured to output a mixture of the blood sample and the reagent; and a helical flow path extending between the inlet and the outlet, wherein the helical mixer is configured to mix the blood sample with the reagent.

Advantageously, one or more example helical mixers can be suitable for microfluidics, such as having one or more structures suitable for microfluidics. The helical mixer can utilize microfluidics, such as for example inertial microfluidics, to, for example, mix the blood sample and the reagent. For example, the helical mixer can form, such as use, such as be suitable to form, counter rotating vortices perpendicular to a main flow path. In one or more example helical mixers, the helical mixer can be configured for microfluidics.

For example, the inlet can be configured to receive a blood sample, which may be the initial blood sample and/or the second blood sample as disclosed herein. Alternatively, the inlet can be configured to receive a different blood sample. All the aforementioned types of blood samples can be encompassed by the disclosure.

An unmixed combination of the blood sample and the reagent can be received by the inlet. A partially mixed combination of the blood sample and the reagent can be received by the inlet. In one or more example helical mixers, the inlet can receive the blood sample and a reagent can be provided separately into the inlet, or other components of the helical mixer. For example, the helical mixer may receive the blood sample and then be configured to add reagent to the blood sample.

The inlet may be an aperture. The inlet may be an inlet end. The inlet may be an inlet section. The inlet may be an inlet portion.

The outlet can be configured to output a mixture of the blood sample and the reagent. The mixture can be the same as the mixture disclosed with respect to the blood analyzer, or may be a different mixture. The mixture may be a mixed combination of blood sample and reagent.

The outlet may be an aperture. The outlet may be an outlet end. The outlet may be an outlet section. The outlet may be an outlet portion.

In one or more example helical mixers, the helical mixer can be configured to receive a blood sample having a volume in the range from 1 μl to 60 μl. For example, the helical mixer can be configured to receive a blood sample having a volume in the range from 5 μl to 30 μl. The helical mixer can be configured to receive a blood sample having a volume of 1 μl, 2 μl, 3 μl, 4 μl, 5 μl or greater, 10 μl or greater, 15 μl or greater, or 20 μl or greater. The helical mixer can be configured to receive a blood sample having a volume of 60 μl or less, 50 μl or less, 40 μl or less, 30 μl or less, or 20 μl or less.

The helical mixer can be configured to receive the blood sample, such as the initial blood sample and/or the second blood sample, having a volume as discussed with respect to the blood analyzer.

In one or more example helical mixers, the helical mixer can be configured to receive a combination of blood sample and reagent having a volume in the range from 1 μl to 60 μl. For example, the helical mixer can be configured to receive a receive a combination of blood sample and reagent having a volume in the range from 5 μl to 30 μl. The helical mixer can be configured to receive a receive a combination of blood sample and reagent having a volume of 1, 2, 3, 4, 5, 10, 15, or 20 μl or greater. The helical mixer can be configured to receive a combination of blood sample and reagent having a volume of 60, 50, 40, 30, or 20 μl or less.

A helical flow path can extend between the inlet and the outlet. For example, the helical flow path can provide communication, such as fluid communication, between the inlet and the outlet. The helical flow path can be a path for allowing liquid to flow through. The helical flow path may be one or more of: a fully helical flow path, a partially helical flow path, a spiral flow path, a coiled flow path, a corkscrew flow path, a spiraling flow path, a turning flow path, a flow path having turns, and a winding flow path. The helical flow path can include one or more intermediate components.

The helical mixer can use air to push the blood sample and the reagent through the helical mixer, such as through the helical flow path. The air can act as a medium to translate the blood sample and the reagent through the helical flow path. The air can apply a force on an end of the combination of the blood sample and the reagent to translate the blood sample and the reagent through the helical flow path.

In one or more example helical mixers, the helical flow path can include at least 5 turns. In one or more example helical mixers, the helical flow path can include at least 10 turns. The helical flow path may include 10-20 turns. The helical flow path may include at least 10, 11, 12, 13, 14, 15, 16, 17, 18, 19, or 20 turns. The helical flow path may include less than 20, 19, 18, 17, or 16 turns.

A turn, such as a winding, can be defined as the helical path extending in 360 degrees. A turn can be defined as the helical flow path starting from a point on a vertical axis and ending at another point on the vertical axis. A turn can be defined as a rotation, such as a full rotation, of the helical flow path. A number of turns can be defined as a number of rotations the helical flow path makes.

In one or more example helical mixers, the helical flow path can have an inner winding diameter in the range from 200 μm-5 mm. In one or more example helical mixers, the helical flow path can have an inner winding diameter in the range from 200 μm-3 mm. The helical flow path can have an inner winding diameter in the range from 2 mm to 4 mm. The helical flow path can have an inner winding diameter in the range from 3 mm to 3.5 mm. The helical flow path can have an inner winding diameter of 0.1 mm, 0.2 mm, 0.3 mm, 0.4 mm, 0.5 mm, 0.6 mm, 0.7 mm, 0.8 mm, 0.9 mm, or 1 mm or greater. The helical flow path can have an inner winding diameter of 10 mm or less, 9 mm or less, 8 mm or less, 7 mm or less, 6 mm or less, 5 mm or less, 4 mm or less, 3 mm or less, 2 mm or less, or 1 mm or less.

In one or more example helical mixers, the helical flow path can have an outer winding diameter in the range from 200 μm-5 mm. The helical flow path can have an outer winding diameter in the range from 1 mm to 4 mm. The helical flow path can have an outer winding diameter in the range from 2 mm to 3.0 mm. The helical flow path can have an outer winding diameter of 1.35 mm. The helical flow path can have an outer winding diameter of 0.1 mm, 0.2 mm, 0.3 mm, 0.4 mm, 0.5 mm, 0.6 mm, 0.7 mm, 0.8 mm, 0.9 mm, or 1 mm or greater. The helical flow path can have an outer winding diameter of 10 mm or less, 9 mm or less, 8 mm or less, 7 mm or less, 6 mm or less, 5 mm or less, 4 mm or less, 3 mm or less, 2 mm or less, or 1 mm or less.

In one or more example helical mixers, the helical flow path can have an inner diameter of between 0.1 and 2.0 mm. The helical flow path can have an inner diameter of 0.1 mm, 0.2 mm, 0.3 mm, 0.4 mm, 0.5 mm, 0.6 mm, 0.7 mm, 0.8 mm, 0.9 mm, 1.0 mm, 1.1 mm, 1.2 mm, 1.3 mm, 1.4 mm, 1.5 mm, 1.6 mm, 1.7 mm, 1.8 mm, 1.9 mm, or 2.0 mm. The helical flow path can have an inner diameter of greater than 0.1 mm, 0.2 mm, 0.3 mm, 0.4 mm, 0.5 mm, 0.6 mm, 0.7 mm, 0.8 mm, 0.9 mm, 1.0 mm, 1.1 mm, 1.2 mm, 1.3 mm, 1.4 mm, 1.5 mm, 1.6 mm, 1.7 mm, 1.8 mm, 1.9 mm, or 2.0 mm. The helical flow path can have an inner diameter of less than 0.1 mm, 0.2 mm, 0.3 mm, 0.4 mm, 0.5 mm, 0.6 mm, 0.7 mm, 0.8 mm, 0.9 mm, 1.0 mm, 1.1 mm, 1.2 mm, 1.3 mm, 1.4 mm, 1.5 mm, 1.6 mm, 1.7 mm, 1.8 mm, 1.9 mm, or 2.0 mm.

In one or more example helical mixers, the helical flow path can have a cross-sectional area of between 0.01 and 15 mm². The helical flow path can have a cross-sectional area of greater than 0.01, 0.05, 0.1, 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, or 12.0 mm². The helical flow path can have a cross-sectional area of less than 0.01, 0.05, 0.1, 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, or 12.0 mm².

In one or more example helical mixers, the helical flow path can have a volume in the range from 100 mm³ to 1000 mm³. The helical flow path can have a volume (in μl or mm³) of greater than 100, 200, 300, 400, 500, 600, 700, 800, 900, or 1000. The helical flow path can have a volume (in μl or mm³) of less than 100, 200, 300, 400, 500, 600, 700, 800, 900, or 1000.

In one or more example helical mixers, the helical flow path can have a circular cross section. The helical flow path may not have a circular cross section. The helical flow path may have a curved cross section. The helical flow path can have an ovaloid cross section. The helical flow path may not have any edges.

In one or more example helical mixers, the helical flow path has a same diameter along an entire length of the helical flow path. The helical flow path may vary in diameter along a length of the helical flow path.

Advantageously, the helical flow path can allow for easier cleaning. For example, unused blood samples and/or reagents can be easily flushed from the helical mixer. The helical flow path may be sized and configured to be flushed.

The helical flow path can allow for formation of Dean vortices according to Dean flow theory. The Dean vortices can advantageously help with mixing of the reagent and the blood sample. A Dean number (Dₑ) can be determined based on the following equation:

$$De = Re * \sqrt{\frac{{D}_{Tube}}{{D}_{Helix}}} \qquad ( 1 )$$

Where Rₑ is a Reynolds number, D_{tube} is a diameter of the tube, and Dₕₑₗᵢₓ is a diameter of the helix. The diameter of the helix can be from a central axis of the helical flow path. The Dean flow speed (U_{De}) can be calculated as:

$${U}_{De} = 1.84 * {10}^{- 4} * {De}^{1.63} ( m / s ) \qquad ( 2 )$$

In other words, the flow speed through the helical mixer may be larger than the Dean flow speed given by (2). The dean flow speed may be indicative of how quickly liquid flows in a circular rotation. The helical mixer can be sized and configured so that the combination of the blood sample and the reagent undergo at least one full rotation in a first direction from the Dean vortices.

The helical flow path can be suitable for forming counter rotating vortices perpendicular to a main flow path of the helical flow path. The helical flow path can be configured to form counter rotating vortices perpendicular to a main flow path of the helical flow path.

In one or more example helical mixers, the helical flow path can have a length in the range from 10 cm to 40 cm, such as in the range from 20 cm to 30 cm. The helical flow path may have a length of 10, 11, 12, 13, 14, 15, 16, 17, 18, 19, 20, 21, 22, 23, 24, 25, 26, 27, 28, 29, or 30 cm. The helical flow path may have a length larger than 30 cm. The helical flow path may include the inlet. The helical flow path may include the outlet. The helical flow path may not include the inlet. The helical flow path may not include the outlet.

A length of the helical flow path can be defined by a length of the combination of blood sample and reagent in the helical flow path. For example, the length of the helical flow path may be at least 1.5, 2.0, 2.5, 3.0, or 3.5× a length of the combination of blood sample and reagent. The length of the helical flow path may be at least 10.0, 9.0, or 8.0× a length of the combination of blood sample and reagent.

For example, a length of the helical flow path can be greater or equal to 2× a length of the combination of blood sample and reagent in the helical flow path. The length of the of the combination of blood sample and reagent can be the volume of the combination of blood sample and reagent divided by the cross-sectional area of the helical flow path.

In one or more example helical mixers, the helical flow path can surround, such as wrap around, wound around, turn around, be centered on, a center rod. The center rod may provide structural support to the helical flow path. The helical mixer may not contain a center rod, and the helical flow path can support itself.

The center rod may be metal. The center rod may be aluminum. The center rod may be a conducting material. The center rod may have a diameter of 3-5 mm, such as 3.3 mm. The center rod may define the inner winding radius of the helical flow path. The center rod and/or the space or volume between the center rod and the helical flow path and/or between windings, may be filled with a heat conducting material, such as a heat conductive paste. The center rod may be coated with a heat conducting material, such as a paste.

Table I illustrates certain non-limiting example dimensional parameters of an example helical mixer.

TABLE I
Helical mixer dimensions

| | Inner helical | | Helical | Volume |
|---|---|---|---|---|
| | flow path | Diameter | flow path | flow |
| | diameter | of helix | length | (μL/ |
| Example | (mm) | (mm) | (cm) | second) |
| A | 0.7-0.9 | 4-6 | 20-30 | 20-30 |
| B | 1.0-1.5 | 3-5 | 15-20 | 25-45 |
| C | 0.5-0.8 | 6-10 | 30-45 | 15-25 |
| D | 1.3-2.0 | 2-4 | 20-30 | 15-30 |
| E | 0.1-0.5 | 2-4 | 15-20 | 20-30 |
| F | 1.5-2.0 | 6-10 | 15-20 | 50-80 |

In one or more example helical mixers, the helical mixer can comprise a temperature controlling element. The center rod may be or comprise the temperature controlling element. The temperature controlling element may be separate from the center rod. The center rod may include, for example, a diode for dissipating heat. The center rod may include, for example, a sensor such as a thermocouple.

For example, the temperature controlling element may be configured to provide heating and/or cooling to the helical flow path. The temperature controlling element can heat and/or cool the blood sample and/or the reagent. The temperature controlling element can act as the incubator as discussed herein, such as having one or more incubation settings. Any and all discussion on the mixer and/or incubator discussed herein can be incorporated into the helical mixer.

The helical flow path can be configured to mix the blood sample, such as the initial blood sample and/or the second blood sample. For example, as the blood sample and reagent flow through the helical flow path, the blood sample and the reagent may mix together. For example, the blood sample and the reagent may mix together without, or with a minimal of, a gradient level of reagent and/or blood sample.

In one or more example helical mixers, the helical mixer can include a tube forming the helical flow path. The tube can be made of a polymer material. The tube may be a, for example, duct, pipe, path, tunnel and/or lumen. In one or more example helical mixers, the polymer material can include fluorinated ethylene propylene (FEP). The tube can be made of an extruded material, such as an extruded polymer. The tube can be made of extruded FEP. The tube can be one or more of nylon, silicone, vinyl, polyethylene, polypropylene, polyvinylchloride, and polyurethane.

The tube can be made of or comprise a metal material. The tube can be made of a mixture of materials. The particular material of the tube is not limiting. The tube can be made of steel. The tube can be made of aluminum. The tube can be made of a biologically inert material.

The helical flow path, such as the inner surface of the tube, may have a roughness Ra of less than 1.0, 0.9, 0.8, 0.7, 0.6, 0.5, 0.4, 0.3, 0.2, or 0.1 μm.

The helical flow path, such as the inner surface of the tube, may have a roughness Rz of less than 2.0, 1.9, 1.8, 1.7, 1.6, 1.5, 1.4, 1.3, 1.2, 1.1, or 1.0 μm.

The helical flow path, such as the tube, may not have a surface charge.

The helical flow path, such as the tube, can have a sidewall thickness of 0.1 mm-0.5 mm. The helical flow path can have a sidewall thickness (in mm) of 0.1, 0.15, 0.2, 0.25, 0.3, 0.35, 0.4, 0.45, or 0.

In one or more example helical mixers/blood analyzers, the helical mixer/blood analyzer can include a flow direction mechanism. The flow direction mechanism can be configured to change a flow direction of the helical mixer. The flow direction mechanism may be incorporated into the helical mixer. The flow direction mechanism may be configured to communicate with the helical mixer. The flow direction mechanism may be separate from the helical mixer. For example, the flow direction mechanism may be part of the blood analyzer. For example, the blood sample and the reagent can move in a first direction from the inlet to the outlet. The flow direction mechanism may change the flow direction to a second direction wherein the blood sample and the reagent move from the outlet towards the inlet. The flow direction mechanism can reverse the flow through the helical mixer, e.g. during a mixing cycle.

The flow direction mechanism may utilize air, such as atmospheric air, to translate blood sample and the reagent through the helical mixer. For example, a pressure gradient can be formed in the surrounding air segments to translate the blood sample and the reagent through the helical mixer. The flow direction mechanism may be a peristaltic pump, or another type of pump. Other gasses can be used as well, and the particular gas is not limiting. The flow direction mechanism may use air at both ends of the combination of the blood sample and the reagent for translation of the blood sample and the reagent through the system.

The flow direction mechanism can change the flow direction through the helical mixer one or more times. For example, the blood sample and the reagent can pass through the helical flow path one or more times, such as 2, 3, 4, 5, 6, 7, 8, 9, or 10 times. This can allow the blood sample and the reagent to mix together.

In one or more example helical mixers, the helical mixer can be configured for a flow rate in the range from 1 μL/s to 50 μL/s. The helical mixer may have or be configured for a flow rate in the range of 10 μL/s to 40 μL/s. The helical mixer may have a flow rate (μL/s) of at least 10, 20, 30, 40, or 50. The helical mixer may have a flow rate (μL/s) of less than 10, 20, 30, 40, or 50. The flow rate can be low enough so that air is not introduced into the combination of blood sample and reagent.

Advantageously, the disclosed helical mixers can be used for mixing the reagent and the blood sample, such as the initial blood sample and/or the second blood sample, without negatively affecting the blood sample. Negative effects can include, for example, clumping or clotting of the blood sample and/or changing the viscosity of the blood sample.

In one or more example helical mixers, the helical mixer can be configured to not separate particles of the blood sample. The helical mixer can be configured to minimize and/or mitigate separation of particles in the blood sample. As it can be advantageous to analyze the blood sample as it would be in a patient, the helical mixer may not separate different components of the blood sample, such as would occur in a centrifuge. For example, inertial focusing can be avoided and/or minimized. For example, demixing can be avoided. For example, up concentrations can be avoided.

The helical mixer can be configured to not separate any components of the blood sample and reagent. The helical mixer can be configured to minimize and/or mitigate separation of any components of the blood sample and reagent. For example, the helical mixer can be configured so that any subvolume of the mixture of blood sample and reagent can be the same, or generally the same, as any other subvolume. The helical mixer can be configured to form a homogenous mixture of the blood sample and the reagent. The helical mixer can be configured to form a uniform mixture of the blood sample and the reagent. The helical mixer can be configured to form a mixture of the blood sample and the reagent without, or with minimized, gradients.

In one or more example helical mixers, the helical mixer can be configured to not activate platelets and/or white blood cells in the blood sample. The helical mixer can be configured to minimize and/or mitigate activation of platelets and/or white blood cells in the blood sample. For example, the helical mixer will not cause the platelets and/or the white blood cells to take any action that they would normally take in the body against harm or invasive entities.

In one or more example helical mixers, the helical mixer can be configured to mix blood with a reagent without activating platelets in the blood sample.

The helical mixer may be configured to apply a shear force to the blood sample and/or the reagent. For example, the shear force (N/m²) may be 2.0, 1.9, 1.8, 1.7, 1.6, 1.5, 1.4, 1.3, 1.2, 1.1, 1.0, 0.9, 0.8, 0.7, 0.6, 0.5, 0.4, 0.3, 0.2, or 0.1 or less. The shear force may be less than an activation level of the blood sample, such as less than 1.5 N/m² or less than 1.0 N/m².

The helical mixer may be configured for continuous flow. The helical mixer may be configured for non-continuous flow. The helical mixer may be configured for both continuous flow and non-continuous flow.

The helical mixer may include one or more sensors. The one or more sensors may be liquid sensors. The one or more sensors can be configured to determine whether the reagent and blood sample has mixed. The one or more sensors can be located at the outlet. The one or more sensors can be located at the inlet. The one or more sensors can be located in the helical flow path.

The disclosure herein, such as for example with respect to the blood analyzer and/or the helical mixer, will be understood as relating to methods as well.

In one or more example methods, a method of preparing a blood sample is disclosed. The method can include providing a blood sample and a reagent into an inlet. The method can include mixing the blood sample and the reagent via translating the blood sample and the reagent through a helical flow path in communication with the inlet. The method can include outputting the mixed blood sample and the reagent at an outlet in communication with the inlet. The method may be performed using a helical mixer as disclosed herein.

In one or more example methods, mixing can include mixing the blood sample and the reagent without activating platelets and/or white blood cells in the blood sample.

In one or more example methods, mixing may not separate particles of the blood sample. The method can be configured to minimize and/or mitigate separation of particles in the blood sample.

In one or more example methods, mixing can include translating the blood sample and the reagent through the helical flow path in a first direction. In one or more example methods, mixing can include translating the blood sample and the reagent through the helical flow path in a second direction, wherein the second direction is opposite the first direction.

In one or more example methods, translating can be at a flow rate in the range from 1 μL/s to 50 μL/s.

In one or more example methods, translating the blood sample and the reagent through the helical flow path creates a shear force of less than 1.5 N/m².

In one or more example blood analyzers, the blood count sensor device can further include an incubator, such as for heating/incubating the initial blood sample and/or the second blood sample and the reagent and/or the mixture. The incubator may be configured to heat a portion of the fluid pathway, such as the initial fluid pathway and/or the second fluid pathway, thus heating the fluid pathway, such as the initial fluid pathway and/or the second fluid pathway. The incubator may be located after the mixer. The incubator may be located prior to the mixer.

The incubator may include one or more incubation settings. An incubation setting can include one or more parameters of an incubator.

For example, this can include an incubation temperature. In one or more example blood analyzers, the initial blood sample and/or the second blood sample and reagent and/or the mixture may be incubated at a temperature of 30, 40, 45, 46, 47, 48, 49, 50, or 55° C. In one or more example blood analyzers, the initial blood sample and/or the second blood sample and reagent and/or the mixture may be incubated at a temperature of greater than 30, 40, 45, 46, 47, 48, 49, 50, or 55° C. In one or more example blood analyzers, the initial blood sample and/or the second blood sample and reagent and/or the mixture may be incubated at a temperature of less than 30, 40, 45, 46, 47, 48, 49, 50, or 55° C.

The incubation settings can include an incubation time. In one or more example blood analyzers, the initial blood sample and/or the second blood sample and reagent and/or the mixture may be incubated for 1, 2, 3, 4, 5, 6, 7, 8, 9, 10, 11, 12, 13, 14, 15, 16, 17, 18, 19, 20, 21, 22, 23, 24, or 25 seconds. In one or more example blood analyzers, the initial blood sample and/or the second blood sample and reagent and/or the mixture may be incubated for greater than 1, 2, 3, 4, 5, 6, 7, 8, 9, 10, 11, 12, 13, 14, 15, 16, 17, 18, 19, 20, 21, 22, 23, 24, or 25 seconds. In one or more example blood analyzers, the initial blood sample and/or the second blood sample and reagent and/or the mixture may be incubated for less than 1, 2, 3, 4, 5, 6, 7, 8, 9, 10, 11, 12, 13, 14, 15, 16, 17, 18, 19, 20, 21, 22, 23, 24, or 25 seconds. In one or more example blood analyzers, the initial blood sample and/or the second blood sample and reagent and/or the mixture may not be mixing, e.g. may remain stationary, during the incubation. Alternatively, the initial blood sample and/or the second blood sample and reagent and/or the mixture may be moving during incubation. The incubation time may vary based on the incubation temperature.

In one or more example blood analyzers, the blood count sensor device can further include a cooling unit or module, e.g. chiller, cooler, cooling module, such as for cooling the initial blood sample and/or the second blood sample and the reagent and/or the mixture. In particular, the cooling unit can be configured to stop or at least slow down or reduce any lysing and/or staining reactions via the reagent. In particular, the cooling unit can be located after the incubator. The cooling unit may be located before or after the second sensor. The cooling unit may be located after the mixer. The cooling unit may be located between the image sensor and the mixer/incubator.

In one or more example blood analyzers, the cooling unit can be separate from the mixer and/or the incubator. In one or more example blood analyzers, the cooling unit can be a component of the mixer. In one or more example blood analyzers, the cooling unit can be a component of the incubator. In one or more example blood analyzers, the cooling unit can be a component of the mixer-incubator.

In one or more example blood analyzers, the cooling unit can be integrated further in the fluid pathway, such as with respect to the image sensor discussed below. For example, the measurement chamber can be configured to act as the cooling unit.

The cooling unit may be configured to cool a portion of the fluid pathway, such as the initial fluid pathway and/or the second fluid pathway, thus cooling the fluid pathway, such as the initial fluid pathway and/or the second fluid pathway.

The cooling unit may include one or more cooling unit settings. A cooling unit setting can include one or more parameters of a cooling unit.

For example, this can include a cooling temperature. In one or more example blood analyzers, the initial blood sample and/or the second blood sample and reagent and/or the mixture may be cooled at a temperature of 0, 5, 10, 15, 20, 25, 30, 35, 40, 45, or 50° C. In one or more blood example analyzers, the initial blood sample and/or the second blood sample and reagent and/or the mixture may be cooled at a temperature of greater than 0, 5, 10, 15, 20, 25, 30, 35, 40, 45, or 50° C. In one or more example blood analyzers, the initial blood sample and/or the second blood sample and reagent and/or the mixture may be cooled at a temperature of less than 0, 5, 10, 15, 20, 25, 30, 35, 40, 45, or 50° C. The cooling temperature may be less than the incubation temperature. In one or more example blood analyzers, a temperature difference between an incubation temperature and a cooling temperature is at least 10° C., such as at least 20° C.

The cooling unit settings can include a cooling time. In one or more example blood analyzers, the initial blood sample and/or the second blood sample and reagent and/or the mixture may be cooled for 10, 11, 12, 13, 14, 15, 16, 17, 18, 19, 20, 21, 22, 23, 24, or 25 seconds. In one or more example blood analyzers, the initial blood sample and/or the second blood sample and reagent and/or the mixture may be cooled for greater than 10, 11, 12, 13, 14, 15, 16, 17, 18, 19, 20, 21, 22, 23, 24, or 25 seconds. In one or more example blood analyzers, the initial blood sample and/or the second blood sample and reagent and/or the mixture may be cooled for less than 10, 11, 12, 13, 14, 15, 16, 17, 18, 19, 20, 21, 22, 23, 24, or 25 seconds.

In one or more example blood analyzers, the mixer may also be an incubator, such as a mixer-incubator. The incubator can be a component of the mixer. Thus, the mixer can be configured to incubate and mix the initial blood sample and/or the second blood sample and reagent and/or the mixture.

The blood count sensor device may include a blood count sensor, such as a liquid sensor. The blood count sensor may detect when the initial blood sample and/or the second blood sample and reagent and/or the mixture passes through the mixer and/or incubator and/or mixer-incubator.

In one or more example blood analyzers, the blood count sensor device can include a second sensor, e.g. detector, electronic, associated with the fluid pathway, such as the initial fluid pathway and/or the second fluid pathway. In one or more example blood analyzers, the second sensor may not be used.

The second sensor may be associated with the mixer and/or the mixer-incubator and/or the incubator and/or the blood count sensor device and/or the fluid pathway, such as the initial fluid pathway and/or the second fluid pathway. The second sensor can be one or more sensors. The second sensor may be along the fluid pathway, such as the initial fluid pathway and/or the second fluid pathway, in order to obtain sensor data while the blood sample, such as the initial blood sample and/or the second blood sample, passes through the second sensor. The second sensor may determine and/or output data indicative of one or more blood parameters, such as a second blood parameter as discussed below. The second sensor may also be configured to capture an image and provide image data. The terms image data and sensor data may be used interchangeably herein. The second sensor may output the sensor data.

The second sensor may be an image capturer. The second first sensor may be a camera. The second sensor may be an imaging system. The second sensor may be a turbidity sensor. The second sensor may be a liquid sensor. The second sensor maybe a modified liquid sensor. The second sensor may be a porous mirror. The second sensor may be a red blood cell parameter sensor. The first sensor may be a spectrophotometer. The second sensor may be an electronic/second measuring unit. The second sensor may be an optical diffraction tomography unit. The second sensor may be a transmittance sensor. The second sensor may be a refractive index sensor. The second sensor may be a hematocrit (Hct) sensor. The Hct sensor may be used to adapt dosing volume, such as for low Hct samples.

The second sensor may be a hemolysis detector. The hemolysis detector can be sensitive to refraction index changes from lipids. Thus, the hemolysis detector could detect lipid levels.

As mentioned, the second sensor may be an impedance sensor. The impedance sensor may provide sensor data, which may be indicative of the second blood parameter. The impedance sensor may provide a second blood parameter indicative of electrolyte content. The impedance sensor may provide a second blood parameter indicative of lipid content. The impedance sensor may provide a second blood parameter indicative of hematocrit content. The impedance sensor may provide a second blood parameter indicative of electrolyte content and/or lipid content and/or hematocrit content.

The second sensor may be configured to sense one or more electrical and/or optical properties.

In one or more example blood analyzers, the second sensor may be configured to monitor red blood cell lysis. For example, the second sensor may be light absorbance based. Alternatively, the second sensor may be a coulter counter or downwelling light sensor (DLS).

In one or more example blood analyzers, the second sensor may be configured for performing a conductivity analysis. The second sensor can determine the hematocrit, e.g. the volume percentage of red blood cells. For example, electrodes can be used to measure the conductivity. Osmolality can be measured/estimated from the conductivity. The electrodes can span the fluid pathway, such as the initial fluid pathway and/or the second fluid pathway. The electrodes can be incorporated into the fluid pathway, such as the initial fluid pathway and/or the second fluid pathway.

The second sensor may be an absorbance sensor. Thus, the second sensor can perform a light intensity analysis e.g. at one or more different wavelengths. For example, an optical sensor can be used. The optical sensor and/or light source can be at 509, 522, 549, 569, 587, 650, 660, 670, 680, or 690 nm.

In one or more example blood analyzers, the second sensor can be configured to perform a viscosity analysis of the initial blood sample and/or the second blood sample.

In one or more example blood analyzers, the second sensor can be configured to perform a pH analysis of the initial blood sample and/or the second blood sample.

In one or more example blood analyzers, the second sensor can be configured to perform a hematocrit analysis.

In one or more example blood analyzers, the second sensor may obtain the second blood parameter directly. In one or more example blood analyzers, the second sensor may obtain the second blood parameter indirectly. For example, the second sensor may measure a sum of electrolytes and similar analytes of the osmolality, which can affect the ability to lyse and stain cells.

In one or more example blood analyzers, multiple sensors and/or image capturers may be used. Thus, multiple different parameters can be obtained from the initial blood sample and/or the second blood sample. Alternatively, multiple sensors and/or image capturers may be used to obtain and verify the same blood parameter.

As mentioned, the second sensor may be configured to monitor the fluid pathway, such as the initial fluid pathway and/or the second fluid pathway, and provide sensor data. The sensor data may include, directly or indirectly, a second blood parameter. The second blood parameter may be any number of parameters of the initial blood sample and/or the second blood component. In one or more example blood analyzers, the second blood parameter may be a red blood cell content. In one or more example blood analyzers, the second blood parameter may be a plasma content. In one or more example blood analyzers, the second blood parameter may be a lipid content. For example, the lipid content may be from a sample quality index called HIL index. In one or more example blood analyzers, the second blood parameter may be a white blood cell content. In one or more example blood analyzers, the second blood parameter may be a blood gas content. In one or more example blood analyzers, the second blood parameter may be a platelet content. In one or more example blood analyzers, the second blood parameter may be a protein content. In one or more example blood analyzers, the second blood parameter may be a cholesterol content. In one or more example blood analyzers, the second blood parameter may be total cholesterol content. In one or more example blood analyzers, the second blood parameter may be LDL content. In one or more example blood analyzers, the second blood parameter may be LDH content. In one or more example blood analyzers, the second blood parameter may be triglyceride content. In one or more example blood analyzers, the second blood parameter may be a hematocrit content. In one or more example blood analyzers, the second blood parameter may be glucose content. In one or more example blood analyzers, the second blood parameter may be electrolyte content. The second blood parameter may be more than one of the parameters discussed herein, e.g. a combination of two or more. Other second blood parameters can be used as well, and the particular blood parameter is not limiting.

Thus, the second sensor may be configured to detected and/or analyze any of the above second blood parameters. The second sensor may be configured to detect a plurality of the above second blood parameters.

The second sensor may be configured to provide and/or output the sensor data. For example, the second sensor may output the sensor data to a computer system, local and/or remote, as discussed in detail below. The second sensor may output the sensor data to a visual device for review by a user.

In one or more example blood analyzers, the first sensor and the second blood sensor may be the same type of sensor. In one or more example blood analyzers, the first sensor and the second blood sensor may be different types of sensors.

In one or more example blood analyzers, the first parameter and the second blood parameter may be the same. Thus, a user can check the same parameter at separate places in the analyzer. In one or more example blood analyzers, the first parameter and the second blood parameter may be different.

Following the second sensor, the blood count sensor may include an image sensor, e.g. imager, image mechanism, image device, image system, image module. The image sensor may include a microscope. The image system may include a camera. A camera may be combined with other components of the image sensor. The image sensor may be a digital image sensor. The image sensor may be configured to provide image information, e.g. image data. The image sensor may be configured to provide the blood count analysis parameter. The image sensor may be connected to the computer system discussed herein. The image sensor itself may include processors.

In particular, the blood count sensor device may be configured to receive the mixture, so as to be imaged by the image sensor. The blood count sensor device, and thus image sensor, may be in direct fluid communication with the inlet module. The blood count sensor device, and thus image sensor, may be configured to receive an intermediate component for imaging. The blood count sensor device, and thus image sensor, may be configured to accommodate a multi-use cuvette. The blood count sensor device, and thus image sensor, may be configured to accommodate a single-use cuvette.

In one or more example blood analyzers, if a camera were used it may have a pixel resolution of 0.1, 0.2, 0.3, 0.4, 0.5, 0.6, 0.7, 0.8, 0.9, or 1.0 μm. In one or more example blood analyzers, if a camera were used it may have a pixel resolution of greater than 0.1, 0.2, 0.3, 0.4, 0.5, 0.6, 0.7, 0.8, 0.9, or 1.0 μm. In one or more example blood analyzers, if a camera were used it may have a pixel resolution of less than 0.1, 0.2, 0.3, 0.4, 0.5, 0.6, 0.7, 0.8, 0.9, or 1.0 μm.

The image sensor may include one or more filters. For example, the filters may be optical filters. The optical filters may allow for the image sensor to switch optical changes. The optical filters may be used to ensure a monochrome system. Filters may not be used.

The blood count sensor may include a measurement chamber to receive the mixture. The measurement chamber may have a height of 50, 100, 150, 200, 250, 300 μm. The measurement chamber may have a height of less than 50, 100, 150, 200, 250, 300 μm. The measurement chamber may have a height of greater than 50, 100, 150, 200, 250, 300 μm. The particular dimensions are not limiting.

The measurement chamber may have a volume of 0.05, 0.10, 0.15, 0.20, 0.30, 0.40, 0.50, 1.00, 1.50, 2.00, 3.00, 4.00, 5.00, 6.00, 7.00, 8.00, 9.00, or 10.00 μL. The measurement chamber may have a volume of greater than 0.05, 0.10, 0.15, 0.20, 0.30, 0.40, 0.50, 1.00, 1.50, 2.00, 3.00, 4.00, 5.00, 6.00, 7.00, 8.00, 9.00, or 10.00 μL. The measurement chamber may have a volume of less than 0.05, 0.10, 0.15, 0.20, 0.30, 0.40, 0.50, 1.00, 1.50, 2.00, 3.00, 4.00, 5.00, 6.00, 7.00, 8.00, 9.00, or 10.00 μL.

The measurement chamber may advantageously have known dimensions, volumes, and optical qualities. Therefore, the measurement chamber may for example always receive a same volume of the mixture. Therefore, the measurement chamber may have a well-defined area with a well-defined optical path for analysis of the mixture to determine the blood count analysis parameter. As mentioned above, the measurement chamber may be configured to act as a cooling unit.

In one or more example blood analyzers, the blood count sensor may include an illuminator, e.g. illumination device, illumination system, illumination mechanism, light source, radiation source. The illuminator ay be configured to illuminate, e.g. transmit light, transmit radiation, to the measurement chamber. The illuminator may provide Köhler illumination. The particular illumination is not limiting. The illumination may be performed by a lamp and/or light and/or LED and/or filament. For example, a white or monochrome LED may be used. Alternatively, multiple switchable monochrome LEDs may be used having different colors. Further, one or more optical diffusors may be incorporated.

Optical focusing of the measurement chamber may be used to analyze the mixture. For example, an optical focus can be used. The optical focus can be mechanically moved. Alternatively, a digital focus may be used.

The focus may occur through steps, such as measurement steps. The steps can be 1, 2, 3, 4, 5, 6, 7, 8, 9, or 10 μm. The steps can be greater than 1, 2, 3, 4, 5, 6, 7, 8, 9, or 10 μm. The steps can be less than 1, 2, 3, 4, 5, 6, 7, 8, 9, or 10 μm.

In one or more example blood analyzers, chamber height calibration may be performed to properly analyze the mixture. In one or more example blood analyzers, monochrome and/or multiple optical channels may be used.

In one or more example blood analyzers, the blood count sensor device can provide the blood count analysis parameter in less than 180, 170, 160, 150, 140, 130, 120, 110, 100, 90, 80, 70, 60, 50, 40, 30, or 20 seconds from receiving the initial blood sample in the inlet module. In one or more example blood analyzers, the blood count sensor device can provide the blood count analysis parameter in greater than 180, 170, 160, 150, 140, 130, 120, 110, 100, 90, 80, 70, 60, 50, 40, 30, or 20 seconds from receiving the initial blood sample in the inlet module.

The blood count sensor may further include a valve. The valve may fix the mixture in place during measurement, such as the image measurement. The valve may be released to release the mixture from the measurement chamber. For example, the mixture may be released after the blood count analysis parameter is determined.

Moving past the blood count sensor device, in a parallel configuration of the blood count sensor device and the blood gas sensor device the fluid pathways, for example the first fluid pathway and the second fluid pathway, may reconnect and lead to a waste container. Alternatively, the first fluid pathway can lead to a first waste container and the second fluid pathway can lead to a second waste container. One or multiple pumps can be used to move between the first fluid pathway and/or the second fluid pathway and the waste container. For example, one or more peristaltic pumps can be used.

In one or more example blood analyzers, the blood analyzer may include a white blood count sensor. This sensor may measure an absolute count of white blood cells and/or platelets. The white blood count sensor can be located anywhere in the blood analyzer, such as anywhere between the inlet and the waste container.

As detailed above, the blood gas sensor device and the blood count sensor device may be arranged in parallel. Thus, the blood gas analysis parameter and the blood count parameter can be determined in parallel.

Alternatively, the blood gas sensor device and the blood count sensor device may be arranged in series. As the addition of reagent may negatively affect the blood gas sensor device, the initial blood sample may pass through the blood gas sensor device first. The initial blood sample may then continue to the blood count sensor where reagent can be added. As the blood gas sensor device may not use any further chemistry, wet and/or dry, it may not affect the results of the blood count sensor device.

As discussed in detail above, the mixture may be prepared prior to proceeding to the image sensor for determination of the blood count analysis parameter. Specifically, the blood sample, such as the initial blood sample and/or the second blood sample, may include a reagent and/or may be mixed and/or may be incubated and/or may be cooled. The preparation may be based on a preparation scheme and/or a combination scheme. The computer system, discussed below, may be configured to operate the preparation scheme and/or the combination scheme.

The initial addition of reagent from the reagent module may be performed based on a preparation scheme. The preparation scheme can be based on the first blood parameter, such as received from the first sensor and/or the blood gas sensor device. Thus, the preparation scheme may vary based on the first blood parameter. Different preparation schemes can include different preparation settings as discussed below, which may be relevant to the first blood parameter. Thus, advantageously, one or more of the example analyzers can focus the preparation settings to properly adjust the preparation settings for the particular parameter(s) of the blood sample, such as the initial blood sample and/or the second blood sample. This can lead to accurate and fast hematological results such as the blood count analysis parameter.

The preparation scheme may include one or more settings. The preparation scheme may include a mixing setting and/or an incubation setting and/or a cooling setting, all of which are discussed in detail above. Further, the preparation scheme may include a volume setting, discussed below. The preparation scheme may include a volume setting and a mixing setting. The preparation scheme may include a volume setting and an incubation setting. The preparation scheme may include a mixing setting and an incubation setting. The preparation scheme may include an incubation setting and a cooling setting. The preparation scheme may include a mixing setting and an incubation setting and a cooling setting. The preparation scheme may include a volume setting and a mixing setting and an incubation setting and a cooling setting. The preparation scheme can include any number of settings for different modules and/or components and/or methodologies as would be advantageous.

The preparation scheme may be selected, e.g. determined, from a list, e.g. group, table, of different preparation schemes. For example, a preparations scheme can be selected from the list based on the particular first blood parameter.

In one or more example blood analyzers, the list can include a plurality, 3, 4, 5, 10, 15, 20, 25, 30, 35, 40, 50, 60, 70, 80, 90, or 100 different preparation schemes. In one or more example blood analyzers, the list can include greater than 5, 10, 15, 20, 25, 30, 35, 40, 50, 60, 70, 80, 90, or 100 different preparation schemes. In one or more example blood analyzers, the list can include less than 5, 10, 15, 20, 25, 30, 35, 40, 50, 60, 70, 80, 90, or 100 different preparation schemes. The preparation schemes may be stored, such as in the computer system and/or the blood analyzer and/or the blood count sensor device.

In one or more example blood analyzers, the preparation scheme may include a volume setting, such as a first volume setting and/or a second volume setting and/or a third volume setting, etc. Thus, the preparation scheme can include a first volume setting. In one or more example blood analyzers, the first volume setting can be indicative of a volume of the reagent. In one or more example blood analyzers, the first volume setting can be indicative of a total volume of the reagent. In one or more example blood analyzers, the first volume setting can be indicative of a volume of the reagent as compared to a volume of the blood sample, such as a first volume ratio. Different reagent volumes are discussed above with respect to the reagent. Thus, reagent can be added by the reagent module based on the preparation scheme, including the volume setting.

In one or more example blood analyzers, the preparation scheme may include a mixing setting, such as a first mixing setting and/or a second mixing setting and/or a third mixing setting, etc. Thus, the preparation scheme can include a first mixing setting. In one or more example blood analyzers, the first mixing setting can be indicative of a mixing configuration. For example, it can be indicative of a mixing configuration for a mixing of the blood sample, such as the initial blood sample and/or the second blood sample, and the reagent. The mixing settings are detailed above.

In one or more example blood analyzers, the preparation scheme may include an incubation setting, such as a first incubation setting and/or a second incubation setting and/or a third incubation setting, etc. Thus, the preparation scheme can include a first incubation setting. In one or more example blood analyzers, the first incubation setting can be indicative of an incubation configuration. For example, it can be indicative of an incubation configuration for the blood sample, such as the initial blood sample and/or the second blood sample.

In one or more blood example analyzers, the preparation scheme may include a cooling setting, such as a first cooling setting and/or a second cooling setting and/or a third cooling setting, etc. Thus, the preparation scheme can include a first cooling setting. In one or more example blood analyzers, the first cooling setting can be indicative of a cooling configuration. For example, it can be indicative of a cooling configuration for a cooling of the blood sample, such as the initial blood sample and/or the second blood sample, and the reagent. The cooling settings are detailed above.

In one or more example blood analyzers, the preparation scheme may include a reagent identifier of the reagent from a set of available reagent identifiers. For example, a first reagent may have stronger lysing properties than a second reagent. Thus, if stronger lysing may be advantageous, the first reagent may be identified in the preparation scheme.

Thus, in one or more example blood analyzers, the blood analyzer and/or the computer system and/or the blood count sensor device may determine a preparation scheme for the blood sample, such as the initial blood sample and/or the second blood sample, based on the first blood parameter. The different components/modules, such as the mixer and/or incubator and/or reagent module can prepare the blood sample, such as the initial blood sample and/or the second blood sample, based on the preparation scheme.

The preparation scheme may be used to prepare the mixture, e.g. the mixture of reagent and the initial blood sample and/or the second blood sample.

The mixture may undergo further preparation based on a combination scheme. The combination scheme can be based on the second blood parameter, such as received from the second sensor. Thus, the combination scheme can vary based on the second blood parameter. Accordingly, the blood sample, such as the initial blood sample and/or the second blood sample, can first pass through the mixer and/or the incubator to form the mixture. One or more example blood analyzers may then utilize the combination scheme to further adjust the mixture.

As discussed throughout, a combination scheme may be a mixing scheme and/or a second preparation scheme and/or a preparation scheme and/or a composition scheme and/or a composing scheme. The terms may be used interchangeably. The combination scheme can be used to mix and/or combine and/or incubate two components, for example two liquid components, for example a blood sample and a first reagent and/or a second reagent. The combination scheme may be used for partially and/or fully preparing a blood sample for hematological analysis, for example CBC analysis. Thus, the combination scheme may define a preparation of a blood sample via mixing and/or combining and/or incubating.

Different combination schemes can include different combination settings as discussed below, which may be relevant to the second blood parameter. Thus, advantageously, one or more of the example analyzers can focus the combination settings to properly adjust the combination settings for the particular parameter(s) of the mixture. This can lead to accurate and fast hematological results such as the blood count analysis parameter.

The combination scheme may include one or more settings. The combination scheme may include a mixing setting and/or an incubation setting and/or a volume setting, all of which are discussed in detail above. The combination scheme may include a volume setting and a mixing setting. The combination scheme may include a volume setting and an incubation setting. The combination scheme may include a mixing setting and an incubation setting. The combination scheme may include an incubation setting and a cooling setting. The combination scheme may include a mixing setting and an incubation setting and a cooling setting. The combination scheme may include a volume setting and a mixing setting and an incubation setting and a cooling setting. The combination scheme can include any number of settings for different modules and/or components and/or methodologies as would be advantageous.

The combination scheme may be selected, e.g. determined, from a list, e.g. group, table, of different combination schemes. For example, a combination scheme can be selected from the list based on the particular second blood parameter.

In one or more example blood analyzers, the list can include a plurality, 3, 4, 5, 10, 15, 20, 25, 30, 35, 40, 50, 60, 70, 80, 90, or 100 different combination schemes. In one or more example blood analyzers, the list can include greater than 5, 10, 15, 20, 25, 30, 35, 40, 50, 60, 70, 80, 90, or 100 different combination schemes. In one or more example blood analyzers, the list can include less than 5, 10, 15, 20, 25, 30, 35, 40, 50, 60, 70, 80, 90, or 100 different combination schemes. The combination schemes may be stored, such as in the computer system and/or the blood analyzer and/or the blood count sensor device.

In one or more example blood analyzers, the combination scheme may include a volume setting, such as a first volume setting and/or a second volume setting and/or a third volume setting, etc. Thus, the combination scheme can include a first volume setting. In one or more example blood analyzers, the first volume setting can be indicative of a volume of the reagent. In one or more example blood analyzers, the first volume setting can be indicative of a total volume of the reagent. In one or more example blood analyzers, the first volume setting can be indicative of a volume of the reagent as compared to a volume of the mixture, such as a first volume ratio. Different reagent volumes are discussed above with respect to the reagent.

In one or more example blood analyzers, the combination scheme may include a mixing setting, such as a first mixing setting and/or a second mixing setting and/or a third mixing setting, etc. Thus, the combination scheme can include a first mixing setting. In one or more example blood analyzers, the first mixing setting can be indicative of a mixing configuration. For example, it can be indicative of a mixing configuration for a mixing of the mixture. The mixing settings are detailed above.

In one or more example blood analyzers, the combination scheme may include an incubation setting, such as a first incubation setting and/or a second incubation setting and/or a third incubation setting, etc. Thus, the combination scheme can include a first incubation setting. In one or more example blood analyzers, the first incubation setting can be indicative of an incubation configuration. For example, it can be indicative of an incubation configuration for the mixture.

In one or more example blood analyzers, the combination scheme may include a cooling setting, such as a first cooling setting and/or a second cooling setting and/or a third cooling setting, etc. Thus, the combination scheme can include a first cooling setting. In one or more example blood analyzers, the first cooling setting can be indicative of a cooling configuration. For example, it can be indicative of a cooling configuration for a cooling of the mixture. The mixing settings are detailed above.

In one or more example blood analyzers, the combination scheme may include a reagent identifier of the reagent from a set of available reagent identifiers. For example, a first reagent may have stronger lysing properties than a second reagent. Thus, if stronger lysing may be advantageous, the first reagent may be identified in the preparation scheme.

In one or more example blood analyzers, the blood analyzer and/or the computer system and/or the blood count sensor device may determine a combination scheme for the mixture based on the second blood parameter. The different components/modules, such as the mixer and/or incubator and/or reagent module can prepare the mixture based on the combination scheme.

In one or more example blood analyzers, the preparation scheme may not be used, and the combination scheme may be used. For example, the blood analyzer may not include a first sensor. Alternatively, a user can input into the blood analyzer not to use a preparation scheme. In one or more example blood analyzers, the preparation scheme may be used, and the combination scheme may not be used. For example, the blood analyzer may not include a second sensor. Alternatively, a user can input into the blood analyzer not to use a combination scheme. In one or more example blood analyzers, the preparation scheme may not be used, and the combination scheme may not be used. For example, the blood analyzer may not include a first sensor or a second sensor. Alternatively, a user can input into the blood analyzer not to use a preparation scheme or a combination scheme.

If the preparation scheme is not used, once the blood sample and a volume of the reagent are combined with the blood sample, such as the initial blood sample and/or the second blood sample, the analyzer may proceed via an initial combination scheme. The initial combination scheme may include a mixing setting and/or an incubation setting. Thus, the blood sample, such as the initial blood sample and/or the second blood sample, with the reagent may be mixed and/or incubated and/or cooled to form the mixture.

The initial combination scheme may not be based on any blood sample parameters, such as the first blood parameter and/or second blood parameter as discussed herein. Thus, the initial combination scheme may include a “standard” mixing setting and/or “standard” incubation setting and/or a “standard” volume setting and/or a “standard” cooling setting. Alternatively, a user may choose one or more of a mixing setting and/or an incubation setting and/or a volume setting and/or a cooling setting.

Alternatively, the initial combination scheme may be based on previous blood parameters received, such as the first blood parameter. Thus, the previous blood parameters may be obtained prior to the first blood parameter as discussed herein. The previous blood parameters may have been obtained prior to the combining the blood sample with the first volume of first reagent. Alternatively, the initial combination scheme may be based on standard settings provided by a user.

In one or more example blood analyzers, the initial combination scheme may be the preparation scheme.

In one or more example blood analyzers, the blood analyzer may optionally include further capabilities. For example, the blood analyzer may determine a quality parameter of the mixture.

The quality parameter may be determined via the first sensor and/or the second sensor. The quality parameter may be determined via the image sensor.

The quality parameter may be indicative of one or more properties of the mixture. The quality parameter may be indicative of red blood cell lysing of the mixture. The quality parameter may be indicative remaining red blood cells of the mixture. The quality parameter may be indicative of staining of white blood cells of the mixture. The quality parameter may be indicative of staining of platelets of the mixture.

Further, the blood analyzer can be configured to determine whether the quality parameter satisfies or does not satisfy one or more quality criterion. The quality criterion can be a numerical value of certain threshold parameters of the first mixture. The quality criterion may be one or more values. For example, the quality criterion may be satisfied if the quality parameter is above the quality criterion. The quality criterion may be satisfied if the quality parameter is below the quality criterion. The quality criterion may be satisfied if the quality parameter is between two values of the quality criterion.

The quality criterion may include a first lysis threshold. The quality criterion may be met when a first lysis threshold of 95, 96, 97, 98, 99, 99.5, 99.6, 99.7, 99.8, 99.9, or 100% of red blood cells being lysed is met. For example, the quality criterion may be met at a first lysis threshold of 99.5% of red blood cells being lysed. If the quality parameter of the mixture indicates that 99.6% of red blood cells are lysed, the quality criterion would be satisfied. If 99.4% of red blood cells were lysed, the quality criterion would not be satisfied.

The quality criterion may include a second lysis threshold. In one or more example blood analyzers, the quality criterion may be met when 5, 4, 3, 2, 1, 0.5, 0.4, 0.3, 0.2, 0.1, or 0.0% of white blood cells and/or platelets are lysed. For example, the quality criterion may be met at a first lysis threshold 0.2% of white blood cells and/or platelets being lysed. If the quality parameter of the mixture indicates that 0.3% of white blood cells and/or platelets are lysed, the quality criterion would be satisfied. If 0.1% of white blood cells and/or platelets were lysed, the quality criterion would not be satisfied.

In one or more example methods, the quality criterion may include a third lysis threshold. The third lysis threshold may be indicative of staining. In one or more example methods, the quality criterion may be met when 90, 91, 92, 93, 94, 95, 96, 97 98, or 99% of white blood cells and/or platelets are stained. The quality criterion may be met when greater than 90, 91, 92, 93, 94, 95, 96, 97 98, or 99% of white blood cells and/or platelets are stained. The quality criterion may be met when less than 90, 91, 92, 93, 94, 95, 96, 97 98, or 99% of white blood cells and/or platelets are stained.

In accordance with a determination that the quality criterion is satisfied, the analyzer may allow the mixture to proceed to the image sensor to be analyzed, such as discussed herein.

In accordance with a determination that the quality criterion is not satisfied, the analyzer may reverse fluid flow direction and not allow the mixture to proceed to the image sensor. The analyzer may then be configured to determine a second combination scheme.

The second combination scheme may include any and all of the settings discussed above with regard to the combination scheme and, such as the mixing setting, the volume setting, the cooling setting, and/or the incubation setting.

Thus, the blood analyzer may be configured to add a second volume of reagent to the mixture according to the second combination scheme. Alternatively, the blood analyzer can be configured to add a different reagent to the mixture. The mixture may undergo additional mixing and/or incubation and/or cooling according to the second combination scheme.

Thus, advantageously the blood analyzer can be configured to repeat the reagent adding and/or the mixing and/or the incubating until the quality criterion is met. For example, a second mixture, third mixture, fourth mixture, etc. can be formed and may undergo a respective second combination scheme, third combination scheme, fourth combination scheme, etc. Thus, it will be understood that the second, third, fourth, etc. mixture may include any or all of the discussions relevant to the mixture. Further, it will be understood that the second, third, fourth, etc. combination schemes may include any or all of the discussions relevant to the combination scheme.

Thus, the blood analyzer may be configured to iteratively prepare the mixture. The blood analyzer can be configured to add reagent in small doses to advantageously properly lyse the red blood cells and/or stain the white blood cells and/or platelets without lysing the white blood cells and/or platelets.

In one or more example blood analyzers, the blood count sensor device can initiate the preparation scheme and/or the initial combination scheme. As the blood count sensor device is preparing the mixture, the blood count sensor device may receive a first blood parameter from the blood gas sensor device, such as the blood gas analysis parameter, if the devices are acting in parallel. The first blood parameter and/or the new first blood parameter may indicate that the blood sample is out of scope or cannot be analysed, for example too far outside of a standard blood sample, and/or damaged, such as at least partly clotted, and/or unusable so that the blood count sensor device and/or blood analyzer may abort, e.g. stop, cancel, end, the analysis of the mixture and the mixture may be discarded.

A second initial blood sample may then be used in the inlet module. The initial combination scheme and/or the preparation scheme may be adjusted based on the first blood parameter. The aborting may be performed automatically. The aborting may be indicated to user, such as at the output. The aborting may be performed manually.

In one or more example blood analyzers, the analyzer may further include a computer system, e.g., processor, computer, processing system. The computer system can be configured to receive and/or store and/or process data, such as image or sensor data. The computer system can contain algorithms, such as segmentation and/or artificial neural network (ANN) and/or convolutional neural network (CNN) and/or clustering and/or classification algorithms, such as for image analysis and/or creation of preparation schemes and/or combination schemes. The computer system can be configured to operate the blood gas sensor. The computer system can be configured to operate the blood count sensor. The computer system can be located within the housing. The computer system can be separate, e.g. physically separate, from the housing. The computer system can be partially or completely remote, e.g. physically remote, from the housing.

The computer system can be configured to provide the output and/or be the output and/or be associated with the output. For example, the computer system can include a display. The output can include a display configured to visually provide the blood gas analysis parameter. The output can include a display configured to visually provide the blood count parameter. The output can include a display configured to visually provide the blood gas parameter and the blood count parameter. Alternatively, the computer system may be configured to provide the output directly to a user via another component. For example, the computer system may be wired or wirelessly connected to a cellular phone, tablet, laptop, etc. which can display the output. The output can provide the blood gas analysis parameter and the blood count parameter simultaneously. The output can provide the blood gas analysis parameter and the blood count parameter sequentially.

The computer system may be connected, such as electrically or for providing data, between one or more of the modules discussed above. In one or more example blood analyzers, the computer system can be connected to the first sensor. In one or more example blood analyzers, the computer system can be connected to the second sensor. In one or more example blood analyzers, the computer system can be connected to the mixer. In one or more example blood analyzers, the computer system can be connected to the incubator. In one or more example blood analyzers, the computer system can be connected to the cooling unit. In one or more example blood analyzers, the computer system can be connected to the inlet module. In one or more example blood analyzers, the computer system can be connected to the blood gas sensor device. In one or more example blood analyzers, the computer system can be connected to the blood count sensor device. The computer system may be connected to any or all of the listed modules. The computer system may be wirelessly and/or a wired connected.

The computer system can be configured to operate the blood gas sensor and/or the blood count sensor according to one or more preparation schemes. The computer system can be configured to operate the blood gas sensor according to one or more preparation schemes. The computer system can be configured to operate the blood count sensor according to one or more preparation schemes.

In one or more example blood analyzers, the computer system can be configured to control the first sensor. In one or more example blood analyzers, the computer system can be configured to control the second sensor. In one or more example blood analyzers, the computer system can be configured to control the fluidics, such as the flow direction. In one or more example blood analyzers, the computer system can be configured to control the reagent dosing. In one or more example blood analyzers, the computer system can be configured to control the mixer. In one or more example blood analyzers, the computer system can be configured to control the incubator, such as for incubation time and/or incubation temperature.

In one or more example blood analyzers, the computer system can further include one or more controllers. The controller can be configured to allow a user to operate one or more modules of the analyzer. The controller can allow a user to stop or start any or all of the modules of the analyzer. The controller can allow a user to abort the use of the analyzer. The controller can be a physical controller. The controller may be on and/or associated with the blood analyzer and/or the housing. The controller may be an application on, for example, a laptop and/or computer and/or phone and/or tablet.

Accordingly, the different modules may be operated manually by a user or automatically by the blood analyzer and/or computer system. In one or more example blood analyzers, a user may input the initial blood sample and receive the hematological results without needing to take any further action.

In one or more example blood analyzers, the computer system can include a computer program product. The computer program product can include a non-transitory computer readable medium. The non-transitory computer readable medium can have thereon a computer program. The computer program can include program instructions. The computer program can be loadable into a data processing unit. The computer program can be configured to cause execution of the steps, processes, and/or modules discussed above. For example, when the computer program is run by a data processing unit.

Advantageously, In one or more example blood analyzers, the blood analyzer can provide the blood count analysis parameter and the blood gas analysis parameter in less than 180, 170, 160, 150, 140, 130, 120, 110, 100, 90, 80, 70, 60, 50, 40, 30, 20, 15, 10, or 5 seconds from receiving the initial blood sample in the inlet module. In one or more example blood analyzers, the blood analyzer can provide the blood count analysis parameter and the blood gas analysis parameter in greater than 180, 170, 160, 150, 140, 130, 120, 110, 100, 90, 80, 70, 60, 50, 40, 30, 20, 15, 10, or 5 seconds from receiving the initial blood sample in the inlet module.

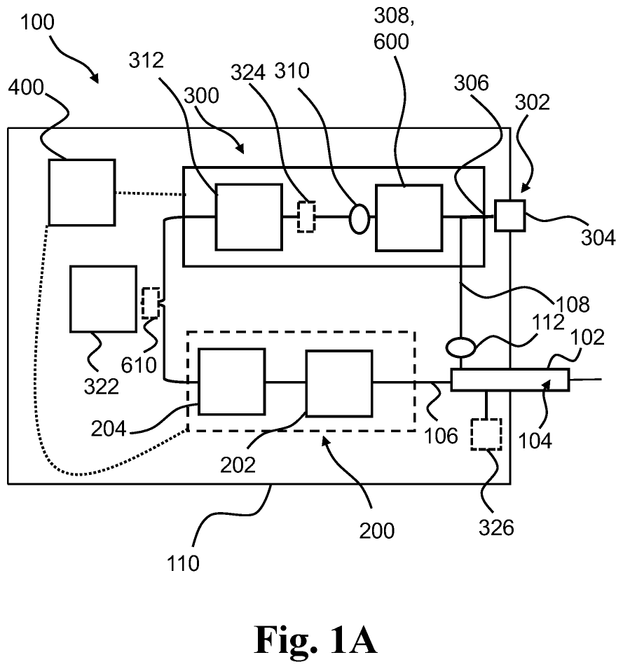

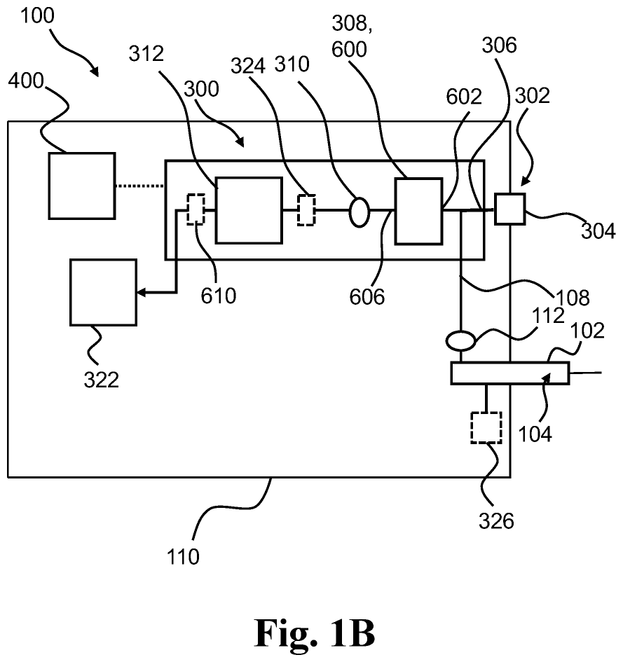

FIG. 1A illustrates a schematic of an example blood analyzer. As shown, the blood analyzer can be a parallel blood analyzer 100. The blood analyzer 100 can include a housing 110, which can contain the modules discussed herein.

The blood analyzer 100 can include an inlet module 102, which can be configured to receive an initial blood sample. The inlet module 102 can provide the initial blood sample to an initial fluid pathway 104. Further, the blood analyzer may include a cleaning module 326 for feeding cleaning fluid to the inlet module 102 for cleaning the fluid/flow paths of the system between test cycles. The initial fluid pathway 104 may include a first sensor 112, which can be configured to determine a first blood parameter. The initial fluid pathway 104 can then split into a first fluid pathway 106 receiving a first blood sample and a second fluid pathway 108 receiving a second blood sample. For example, a divider can be used.

The first fluid pathway 106 can lead to a blood gas sensor device 200. The blood gas sensor device 200 can be in fluid communication with the inlet module 102 and be configured to receive a first blood sample of the initial blood sample and conduct a blood gas analysis on the first blood sample to determine a blood gas analysis parameter. For example, the first blood sample can pass through an oximetry sensor 202 and an electrolyte sensor 204, which can determine the blood gas analysis parameter. Other sensors can be used as well, and the oximetry sensor 202 and the electrolyte sensor 204 are merely examples.

The second fluid pathway 108 can lead to the blood count sensor device 300. The blood count sensor device 300 is in fluid communication with the inlet module 102, and is configured to receive a second blood sample of the initial blood sample and determine a blood count parameter.

As the second blood sample travels down the second fluid pathway 108 it can be combined with a reagent, such as a configured to lyse and stain the second blood sample, from the reagent module 302. The reagent can be located in the reagent reservoir 304 and follow a reagent inlet 306 to mix with the second blood sample. The amount of reagent can be determined via the first blood parameter.

Next, the second blood sample and reagent can pass through a mixer-incubator 308 to form a mixture. The mixture may pass through a second sensor 310 which can determine a second blood parameter. If more reagent is needed based on the second blood parameter, the mixture can pass back through the mixer-incubator 308 to the reagent inlet 306 and more reagent can be added. Further, the mixture may, for example, pass through a cooling unit 324 for cooling the mixture. Once the mixture has been properly prepared, it can pass through an image sensor 312 which can determine a blood count analysis parameter of the mixture.

The first blood sample and the second blood sample can after being analyzed leave their respective blood gas sensor device 200 and blood count sensor device 300 to pass to a waste container 322 for disposal.

As shown, the blood gas sensor device 200 and the blood count sensor device 300 can provide data, including the blood count analysis parameter and blood gas analysis parameter to an output 400. The output 400 may include, or be connected to, a computer system for any processing. The output 400 can be configured to provide the blood gas analysis parameter and/or the blood count parameter.

As shown, the housing 110 accommodates the inlet module 102, the blood gas sensor device 200, the blood count sensor device 300, and the output 400.

FIG. 1B illustrates a schematic of an example blood analyzer as disclosed in FIG. 1A but without blood gas sensor 200.

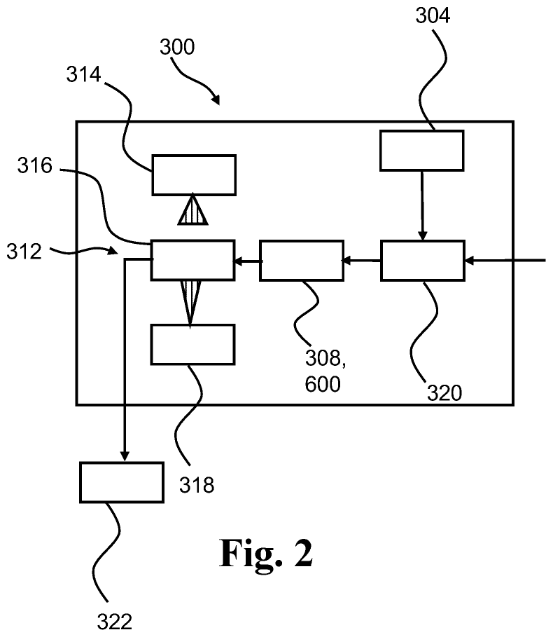

FIG. 2 illustrates a schematic of the blood count sensor device 300. Thus, the blood count sensor device 300 can include any or all of the components discussed above with respect to FIG. 1A and/or FIG. 1B unless specifically mentioned.

As shown, the reagent reservoir 304 may be connected to the second fluid pathway 108 (as illustrated in FIGS. 1A and/or 1B) via a dose valve 320. The dose valve 320 may provide a desired amount of reagent to the second blood sample.

Further, the image sensor 312 can include a number of components. As shown, the image sensor 312 can include a cuvette 316, such as a multi-use cuvette. The cuvette 316 can receive the mixture of the second blood sample and the reagent. The cuvette 316 can be sized for proper image analysis of the mixture.

The image sensor 312 can further include a light source 318 for providing light to the cuvette 316, where it can be analyzed via a camera 314. Image data from the camera can be utilized to determine the blood count analysis parameter. The mixture can after analysis then be discarded into the waste container.

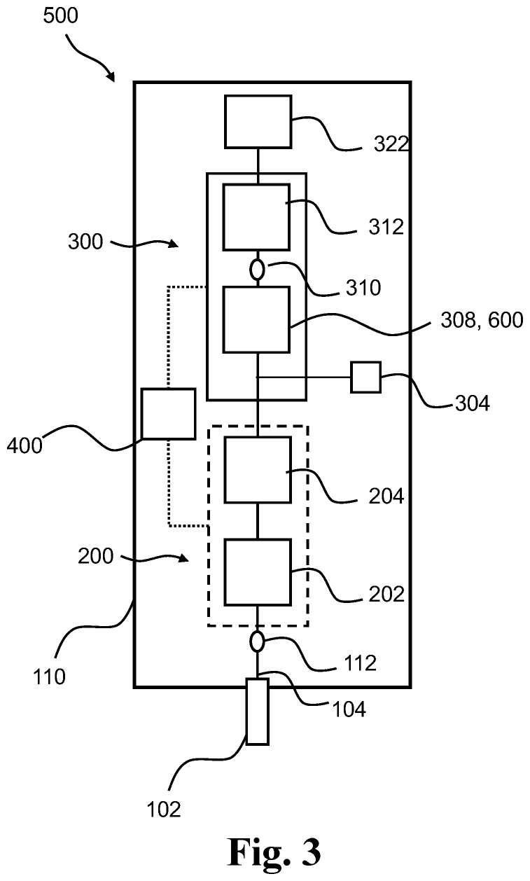

FIG. 3 illustrates an alternate configuration of a blood analyzer. Specifically, FIG. 3 shows a series blood analyzer 500. Blood analyzer 500 can include any or all of the components/modules discussed above with respect to blood analyzer 100 of FIG. 1A and/or FIG. 1B unless specifically mentioned.

As shown, the blood gas sensor device 200 and blood count sensor device 300 are connected in series. Therefore, the initial fluid pathway 104 remains and does not branch off.

The blood gas sensor device 200 is located closer to the inlet module 102 than the blood count sensor device 300. This occurs because the blood gas sensor device 200 does not require any preparation of the initial blood sample, like addition of a reagent. For example, addition of reagent may negatively affect the blood gas analysis parameter.

Once the initial blood sample passes through the blood gas sensor device 200, it enters the blood count sensor device 300. As discussed above, reagent may at this point be added to the initial blood sample and a blood count analysis parameter may be determined. Both the blood gas analysis parameter and the blood count analysis parameter may be provided to the output 400, and the mixture of the initial blood sample and the reagent may be discarded into the waste container 322.

The blood analyzers 100/500 can be used as a point of care system.

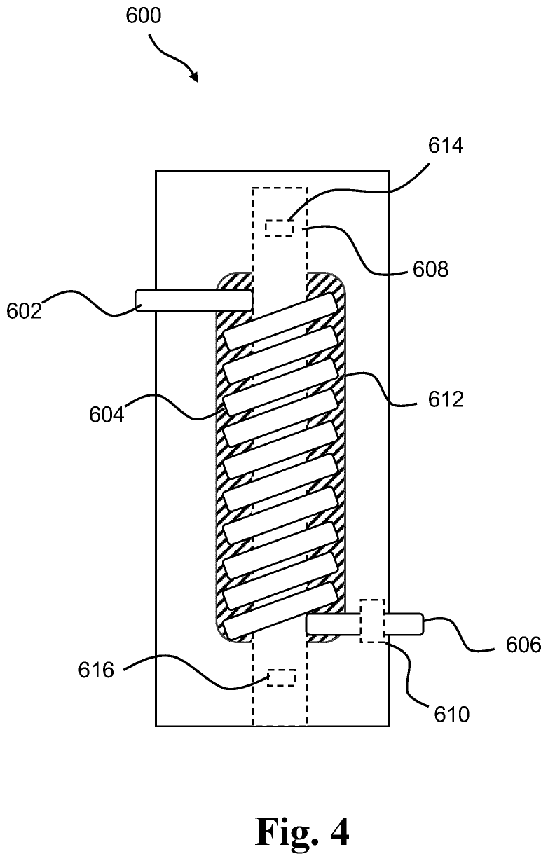

FIG. 4 illustrates an example helical mixer 600 according to the disclosure. As shown, the helical mixer 600 can include an inlet 602. The inlet 602 can lead to a helical flow path 604. The helical flow path 604 can be a tube, such as an FEP tube. The helical flow path 604 can end at an outlet 606.

The helical mixer 600 can receive a blood sample, such as the initial blood sample and/or the second blood sample, and a reagent. The helical mixer 600 can mix the blood sample and the reagent together, such as passing the blood sample and the reagent through the helical flow path 604. The bloods sample and the reagent can be passed through the helical flow path 604 one or more times, such as using optional flow direction mechanism 610. Further, the helical mixer 600 can optionally include a rod, such as a temperature controlling element 608. The windings of the helical flow path 604 surrounds the rod. The temperature controlling element 608 can provide heat and/or cooling to the helical flow path 604. A heat conductive paste 612 may be distributed between and in contact with the windings of the helical flow path 604 and in contact with the heat controlling element 608 for improved heat transfer from/to the temperature controlling element 608 to/from the helical flow path 604. The heat controlling element 608 may optionally include a diode 614, such as for providing heat, and a thermocouple 616, such as for temperature sensing.

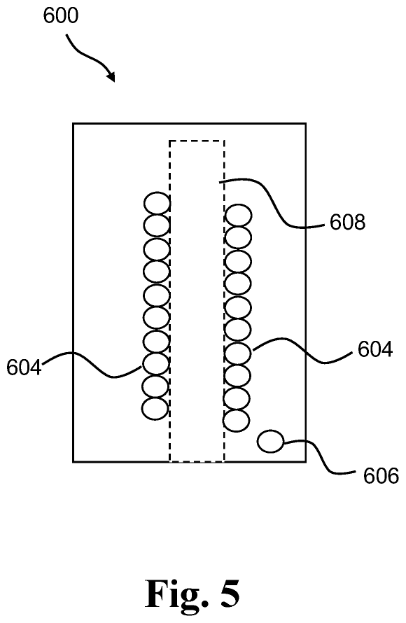

FIG. 5 illustrates a cross-section of the helical mixer 600 shown in FIG. 4.

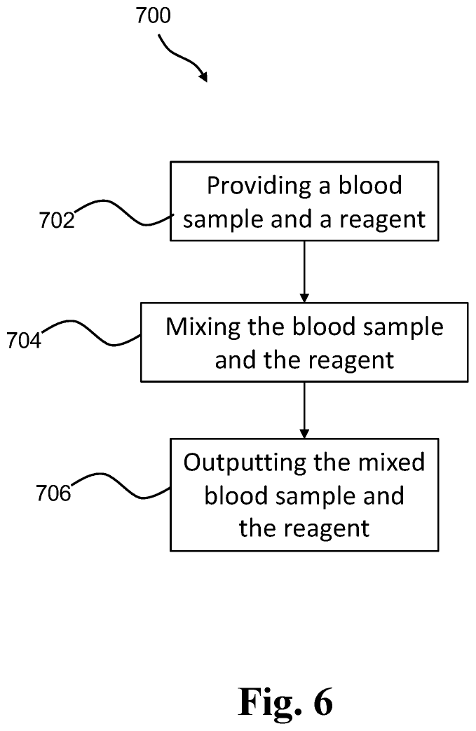

FIG. 6 illustrates a method 700 for preparing a blood sample. The method 700 can include providing 702 a blood sample and a reagent into an inlet. The method 700 can include mixing 704 the blood sample and the reagent via translating the blood sample and the reagent through a helical flow path in communication with the inlet, such as helical flow path described herein. The method 700 can include outputting 706 the mixed blood sample and the reagent at an outlet in communication with the inlet.

In one or more example methods, mixing 704 can include mixing the blood sample and the reagent without activating platelets and/or white blood cells in the blood sample.

In one or more example methods, mixing 704 does not separate particles of the blood sample.

In one or more example methods, mixing 704 includes translating the blood sample and the reagent through the helical flow path in a first direction. In one or more example methods, mixing 704 includes translating the blood sample and the reagent through the helical flow path in a second direction, wherein the second direction is opposite the first direction. For example, mixing 704 may include performing one or a plurality of flow cycles, such as at least 3 flow cycles, wherein each flow cycle comprises translating the blood sample and the reagent through the helical flow path in a first direction, e.g. from the inlet towards the outlet, and translating the blood sample and the reagent through the helical flow path in a second direction, wherein the second direction is opposite the first direction.

In one or more example methods, translating can be at a flow rate in the range from 1 μL/s to 50 μL/s.

In one or more example methods, translating the blood sample and the reagent through the helical flow path can create a shear force of less than 1.5 N/m².

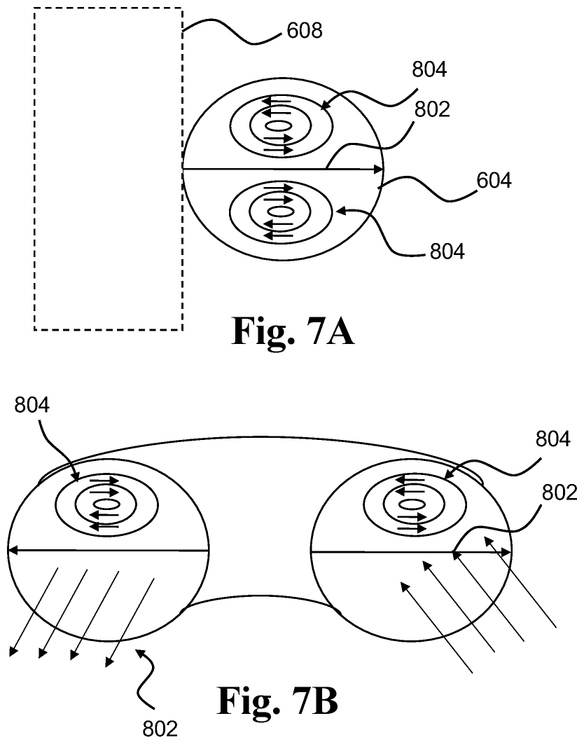

FIGS. 7A-7B illustrate a helical flow path according to a helical mixer as disclosed herein. As shown, the helical flow path 604 can receive a combination of the blood sample and the reagent via a flow path 802 through the helical flow path 604. The helical flow path can be configured so that vortices 804, such as Dean vortices, are formed, thereby mixing the reagent and the blood sample together while translating through the helical flow path 604.

Examples of helical mixers and/or blood analyzers according to the disclosure are set out in the following items:

Item 1. A blood analyzer for analyzing multiple blood parameters, the blood analyzer comprising:
an inlet module configured to receive an initial blood sample;
an optional blood gas sensor device in fluid communication with the inlet module, the blood gas sensor device configured to receive a first blood sample of the initial blood sample and conduct a blood gas analysis on the first blood sample to determine a blood gas analysis parameter;
a blood count sensor device in fluid communication with the inlet module, the blood count sensor device configured to receive a second blood sample of the initial blood sample and determine a blood count parameter; and
an output configured to provide the blood gas analysis parameter and/or the blood count parameter.

Item 2. Blood analyzer according to Item 1, wherein the blood analyzer comprises a housing accommodating the inlet module, the blood gas sensor device, the blood count sensor device, and the output.

Item 3. Blood analyzer according to any of the preceding Items, wherein the blood count sensor device comprises:
a reagent module configured to add a reagent to the second blood sample; and
a mixer configured to mix the reagent with the second blood sample thereby forming a mixture;
wherein the blood count parameter is determined from the mixture.

Item 4. Blood analyzer according to Item 3, wherein the reagent is configured to lyse and/or stain the second blood sample.

Item 5. Blood analyzer according to any one of Item 3 or Item 4, wherein the mixer is a helical mixer.

Item 6. Blood analyzer according to any of the preceding Items, wherein the blood count sensor device is configured to accommodate a multi-use cuvette.

Item 7. Blood analyzer according to any of the preceding Items, wherein the blood analyzer is a point of care system.

Item 8. Blood analyzer according to any of the preceding Items, wherein the initial blood sample comprises an anticoagulant.

Item 9. Blood analyzer according to any of the preceding Items, wherein the blood count sensor device comprises an image sensor.

Item 10. Blood analyzer according to Item 9, wherein the image sensor is a microscope.

Item 11. Blood analyzer according to any of the preceding Items, wherein the first blood sample has a volume less than 100 μL, such as less than 80 μL, or less than 50 μL.

Item 12. Blood analyzer according to any of the preceding Items, wherein the second blood sample has a volume less than 50 μL, such as less than 30 μL, in the range from 10 μL to 25 μL.

Item 13. Blood analyzer according to any of the preceding Items, wherein the initial blood sample is less than 150 μL, such as less than 100 μL.

Item 14. Blood analyzer according to any of the preceding Items, wherein the inlet module comprises a divider configured to divide the initial blood sample into the first blood sample and the second blood sample.

Item 15. Blood analyzer according to any of the preceding Items, wherein the blood gas sensor device and the blood count sensor device are connected to the inlet module in parallel or in series.

Item 16. Blood analyzer according to any one of Items 1-14, wherein the blood gas sensor device and the blood count sensor device are connected in series, and wherein the blood gas sensor device is closer to the inlet module than the blood count sensor device.

Item 17. Blood analyzer according to any of the preceding Items, wherein the blood count sensor device uses wet chemistry or dry chemistry.

Item 18. Blood analyzer according to any of the preceding Items, wherein the output provides the blood gas analysis parameter and the blood count parameter simultaneously.

Item 19. Blood analyzer according to any of the preceding Items, wherein the output provides the blood gas analysis parameter and the blood count parameter within 180 seconds, such as less than 120 seconds, after insertion of the initial blood sample into the inlet module.

Item 20. Blood analyzer according to any of the preceding Items, wherein the blood gas sensor device comprises an oximetry sensor and/or a BMP plate and/or a hemolysis detector and/or an electrolyte sensor.

Item 21. Blood analyzer according to any of the preceding Items, wherein the output comprises a display configured to visually provide the blood gas analysis parameter and the blood count parameters.

Item 22. Blood analyzer according to any of the preceding Items, wherein the analyzer comprises a computer system configured to operate the blood gas sensor and/or the blood count sensor according to one or more preparation schemes.

Item 23. A helical mixer for a blood analyzer, the helical mixer comprising:
an inlet configured to receive a blood sample and a reagent;
an outlet configured to output a mixture of the blood sample and the reagent; and
a helical flow path extending between the inlet and the outlet, wherein the helical mixer is configured to mix the blood sample with the reagent.

Item 24. Helical mixer according to Item 23, wherein the helical flow path has an inner winding diameter in the range from 200 μm-10 mm.

Item 25. Helical mixer according to any one of Items 23-24, wherein the helical mixer is configured to receive the blood sample having a volume in the range from 1 μl to 60 μl.

Item 26. Helical mixer according to any one of Items 23-25, wherein the helical flow path has a circular cross section.

Item 27. Helical mixer according to any one of Items 23-26, wherein the helical flow path has a length in the range from 10 cm to 30 cm.

Item 28. Helical mixer according to any one of Items 23-27, wherein the helical flow path has a same diameter along an entire length of the helical flow path.

Item 29. Helical mixer according to any one of Items 23-28, wherein the helical mixer comprises a tube forming the helical flow path, wherein the tube is made of a polymer material.

Item 30. Helical mixer according to Item 29, wherein the polymer material comprises fluorinated ethylene propylene (FEP).

Item 31. Helical mixer according to any one of Items 23-30, wherein the helical flow path comprises at least 5 turns.

Item 32. Helical mixer according to any one of Items 23-31, wherein the helical mixer further comprises a temperature controlling element.

Item 33. Helical mixer according to any one of Items 23-32, wherein the helical mixer comprises a flow direction mechanism, wherein the flow direction mechanism is configured to change a flow direction of the helical mixer.

Item 34. Helical mixer according to any one of Items 23-33, wherein the helical mixer is configured for a flow rate in the range from 1 μL/s to 50 μL/s.

Item 35. Helical mixer according to any one of Items 23-34, wherein the helical mixer is configured to not separate particles of the blood sample.

Item 36. Helical mixer according to any one of Items 23-35, wherein the helical mixer is configured to not activate platelets and/or white blood cells in the blood sample.

Item 37. Helical mixer according to any one of Items 23-36, wherein the helical mixer is configured to mix blood with a reagent without activating platelets in the blood sample.

Item 38. Helical mixer according to any one of Items 23-37, wherein the helical mixer is configured for microfluidics.

Item 39. A method of preparing a blood sample, the method comprising:
providing a blood sample and a reagent into an inlet;
mixing the blood sample and the reagent via translating the blood sample and the reagent through a helical flow path in communication with the inlet; and
outputting the mixed blood sample and the reagent at an outlet in communication with the inlet.

Item 40. Method of Item 39, wherein mixing comprises mixing the blood sample and the reagent without activating platelets and/or white blood cells in the blood sample.

Item 41. Method of any one of Items 39-40, wherein mixing does not separate particles of the blood sample.

Item 42. Method of any one of Items 39-41, wherein mixing comprises:
translating the blood sample and the reagent through the helical flow path in a first direction; and
translating the blood sample and the reagent through the helical flow path in a second direction, wherein the second direction is opposite the first direction.

Item 43. Method of any one of Items 39-42, wherein translating is at a flow rate in the range from 1 μL/s to 50 μL/s.

Item 44. Method of any one of Items 39-43, wherein translating the blood sample and the reagent through the helical flow path creates a shear force of less than 1.5 N/m².

Item 45. Method of any one of Items 39-44, wherein mixing the blood sample is performed using a helical mixer according to any one of items 23-38.

Item 46. Blood analyzer according to Item 3, wherein the mixer is a helical mixer according to any one of items 23-38.

In a fifth aspect of the invention, the blood analyzer of any of the other aspects disclosed in present disclosure is configured to analyze biological fluids, such as, e.g., human, animal, mammalian blood, and/or cell cultures. Moreover, in the fifth aspect the blood analyzer is substituted by and/or comprises a biological fluid analyzer, such as, e.g., a blood analyzer and/or a cell culture analyzer.

In the fifth aspect, any disclosed blood sample may be substituted by and/or comprise a biological fluid sample, such, e.g., as a human blood sample, an animal blood sample, a mammalian blood sample, and/or a cell culture sample.

In the fifth aspect, any disclosed prepared blood sample may be substituted by and/or comprise a prepared biological fluid sample, such, e.g., as a prepared human blood sample, a prepared animal blood sample, a prepared mammalian blood sample, and/or a prepared cell culture sample.

In the fifth aspect, any disclosed blood parameter may be substituted by and/or comprise a biological fluid parameter, such as human blood parameter, an animal blood parameter, a mammalian blood parameter, and/or a cell culture parameter.

In some embodiments of the fifth aspect, the cell culture comprises a culture of cells derived from multicellular eukaryotes, such as, e.g., mammalian cells, animal cells, and/or human cells. In some embodiments, the cell culture comprises a culture of cells grown from plant tissue culture, fungal culture, and/or microbiological culture (of microbes).

In the fifth aspect, a cell may therefore be a mammalian cell, an animal cell, a human cell, a plant tissue cultured cell, a fungal cultured cell, or a microbiologically cultured cell.

The use of the terms “first”, “second”, “third” and “fourth”, “primary”, “secondary”, “tertiary” etc. does not imply any particular order, but are included to identify individual elements. Moreover, the use of the terms “first”, “second”, “third” and “fourth”, “primary”, “secondary”, “tertiary” etc. does not denote any order or importance, but rather the terms “first”, “second”, “third” and “fourth”, “primary”, “secondary”, “tertiary” etc. are used to distinguish one element from another. Note that the words “first”, “second”, “third” and “fourth”, “primary”, “secondary”, “tertiary” etc. are used here and elsewhere for labelling purposes only and are not intended to denote any specific spatial or temporal ordering.

Furthermore, the labelling of a first element does not imply the presence of a second element and vice versa.

It may be appreciated that FIGS. 1-7B comprise some modules or operations which are illustrated with a solid line and some modules or operations which are illustrated with a dashed line. The modules or operations which are comprised in a solid line are modules or operations which are comprised in the broadest example embodiment. The modules or operations which are comprised in a dashed line are example embodiments which may be comprised in, or a part of, or are further modules or operations which may be taken in addition to the modules or operations of the solid line example embodiments. It should be appreciated that these operations need not be performed in order presented. Furthermore, it should be appreciated that not all of the operations need to be performed. The example operations may be performed in any order and in any combination.

It is to be noted that the word “comprising” does not necessarily exclude the presence of other elements or steps than those listed.

It is to be noted that the words “a” or “an” preceding an element do not exclude the presence of a plurality of such elements.

It should further be noted that any reference signs do not limit the scope of the claims, that the example embodiments may be implemented at least in part by means of both hardware and software, and that several “means”, “units” or “devices” may be represented by the same item of hardware.

The various example methods, devices, and systems described herein are described in the general context of method steps processes, which may be implemented in one aspect by a computer program product, embodied in a computer-readable medium, including computer-executable instructions, such as program code, executed by computers in networked environments. A computer-readable medium may include removable and non-removable storage devices including, but not limited to, Read Only Memory (ROM), Random Access Memory (RAM), compact discs (CDs), digital versatile discs (DVD), etc. Generally, program modules may include routines, programs, objects, components, data structures, etc. that perform specified tasks or implement specific abstract data types. Computer-executable instructions, associated data structures, and program modules represent examples of program code for executing steps of the methods disclosed herein. The particular sequence of such executable instructions or associated data structures represents examples of corresponding acts for implementing the functions described in such steps or processes.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than or equal to 10% of, within less than or equal to 5% of, within less than or equal to 1% of, within less than or equal to 0.1% of, and within less than or equal to 0.01% of the stated amount. If the stated amount is 0 (e.g., none, having no), the above recited ranges can be specific ranges, and not within a particular % of the value. For example, within less than or equal to 10 wt./vol. % of, within less than or equal to 5 wt./vol. % of, within less than or equal to 1 wt./vol. % of, within less than or equal to 0.1 wt./vol. % of, and within less than or equal to 0.01 wt./vol. % of the stated amount.

Although features have been shown and described, it will be understood that they are not intended to limit the claimed invention, and it will be made obvious to those skilled in the art that various changes and modifications may be made without departing from the spirit and scope of the claimed invention. The specification and drawings are, accordingly to be regarded in an illustrative rather than restrictive sense. The claimed invention is intended to cover all alternatives, modifications, and equivalents.

### LIST OF REFERENCES

100 parallel blood analyzer
102 inlet module
104 initial fluid pathway
106 first fluid pathway
108 second fluid pathway
110 housing
112 first sensor
200 blood gas sensor device
202 oximetry sensor
204 electrolyte sensor
300 blood count sensor device
302 reagent module
304 reagent reservoir
306 reagent inlet
308 mixer-incubator
310 second sensor
312 image sensor
314 camera
316 cuvette
318 light source
320 dose valve
322 waste container
324 cooling unit
326 cleaning module
400 output
500 series blood analyzer
600 helical mixer
602 inlet
604 helical flow path
606 outlet
608 temperature controlling element
610 flow direction mechanism
612 heat conductive paste
614 diode
616 thermocouple
700 method of preparing a blood sample
702 providing a blood sample and a reagent into an inlet
704 mixing the blood sample and the reagent
706 outputting the mixed blood sample and the reagent
802 flow path
804 vortices

1. A helical mixer for a blood analyzer, the helical mixer comprising:
an inlet configured to receive a blood sample and a reagent;
an outlet configured to output a mixture of the blood sample and the reagent; and
a helical flow path extending between the inlet and the outlet, wherein the helical mixer is configured to mix the blood sample with the reagent.

2. The helical mixer according to claim 1, wherein the helical flow path has an inner winding diameter in the range from 200 μm to 10 mm.

3. The helical mixer according to claim 1, wherein the helical mixer is configured to receive the blood sample having a volume in the range from 1 μl to 60 μl.

4. The helical mixer according to claim 1, wherein the helical flow path has a circular cross section.

5. The helical mixer according to claim 1, wherein the helical flow path has a length in the range from 10 cm to 30 cm.

6. The helical mixer according to claim 1, wherein the helical flow path has a same diameter along an entire length of the helical flow path.

7. The helical mixer according to claim 1, wherein the helical mixer comprises a tube forming the helical flow path, wherein the tube is made of a polymer material.

8. The helical mixer according to claim 7, wherein the polymer material comprises fluorinated ethylene propylene (FEP).

9. The helical mixer according to claim 1, wherein the helical flow path comprises at least 5 turns.

10. The helical mixer according to claim 1, wherein the helical mixer further comprises a temperature controlling element.

11. The helical mixer according to claim 1, wherein the helical mixer comprises a flow direction mechanism, wherein the flow direction mechanism is configured to change a flow direction of the helical mixer.

12. The helical mixer according to claim 1, wherein the helical mixer is configured for a flow rate in the range from 1 μL/s to 50 μL/s.

13. The helical mixer according to claim 1, wherein the helical mixer is configured to not separate particles of the blood sample.

14. The helical mixer according to claim 1, wherein the helical mixer is configured to not activate platelets and/or white blood cells in the blood sample.

15. The helical mixer according to claim 1, wherein the helical mixer is configured to mix blood with a reagent without activating platelets in the blood sample.

16. The helical mixer according to claim 1, wherein the helical mixer is configured for microfluidics.

17. A method of preparing a blood sample, the method comprising:
providing a blood sample and a reagent into an inlet;
mixing the blood sample and the reagent via translating the blood sample and the reagent through a helical flow path in communication with the inlet; and
outputting the mixed blood sample and the reagent at an outlet in communication with the inlet.

18. The method according to claim 17, wherein the mixing comprises mixing the blood sample and the reagent without activating platelets and/or white blood cells in the blood sample.

19. The method according to claim 17, wherein the mixing does not separate particles of the blood sample.

20. The method according to claim 17, wherein the mixing comprises:
translating the blood sample and the reagent through the helical flow path in a first direction; and
translating the blood sample and the reagent through the helical flow path in a second direction, wherein the second direction is opposite the first direction.

21. The method according to claim 17, wherein the translating is at a flow rate in the range from 1 μL/s to 50 μL/s.

22. The method according to claim 17, wherein the translating the blood sample and the reagent through the helical flow path creates a shear force of less than 1.5 N/m².
